(19) United States
(12) Patent Application Publication
(10) Pub. No.: US 2019/0029592 A1
(43) Pub. Date: Jan. 31, 2019

(54) DEVICE AND METHOD FOR ASSESSING PERIPHERAL EDEMA

(71) Applicant: East Carolina University, Greenville, NC (US)

(72) Inventors: Jason Yao, Greenville, NC (US); Stephanie George, Greenville, NC (US); Sonya Renae Hardin, Greenville, NC (US)

(21) Appl. No.: 16/047,997

(22) Filed: Jul. 27, 2018

Related U.S. Application Data
(60) Provisional application No. 62/538,371, filed on Jul. 28, 2017.

(51) Int. Cl. A61B 5/00

(52) U.S. Cl. CPC A61B 5/4878; A61B 5/0077; A61B 5/443; A61B 5/4842; A61B 5/442; A61B 5/447; A61B 5/7275; A61B 5/445

(57) ABSTRACT

A method of measuring edema (e.g., peripheral edema and/or lymphedema) is described. The method involves directing a pulse of compressed air or other gas at a skin surface and determining the level of edema using processed camera images of the skin surface after indentation with the compressed air or other gas. The method can be completed within minutes, is simple to perform, and is free of user bias. Devices and systems for measuring edema and/or one or more skin-related properties are also described. The devices can be portable and suitable for use in medical or veterinary settings as well as for in-home/personal use.

### RELATED APPLICATIONS

This application is based on and claims the benefit of U.S. Provisional Patent Application Ser. No. 62/538,371, filed Jul. 28, 2017, the disclosure of which is incorporated herein by reference in its entirety.

### TECHNICAL FIELD

The presently disclosed subject matter relates to methods, devices, and systems for measuring and/or monitoring edema (e.g., peripheral edema and/or lymphedema) and various skin-related properties. The methods, devices and systems can provide rapid, repeatable, non-subjective assessment of edema (and/or skin-related properties) in home, office, clinic, and/or hospital settings.

### BACKGROUND

Peripheral edema is a phenomenon in which there is an abnormal infiltration and excess accumulation of serous fluid in connective tissue and/or in a serous cavity in one or more of the body's extremities (e.g., the feet, hands, ankles, calves, wrists, arms). Peripheral edema can be benign and can correct itself in certain circumstances. However, it can also be an indication of a variety of diseases, such as congestive heart failure (CHF), liver disease (e.g., cirrhosis), kidney disease, lymphedema, hypoalbumenia, and chronic venous insufficiency. For example, in CHF, the presence of edema in the extremities (e.g., the lower extremities) can be a valuable diagnostic marker for the presence of disease. In addition, the progression of the edemic state can be monitored over time and related to the progression of the disease. Peripheral edema can also be the result of too much salt in the diet, allergic reactions, pregnancy, burns (e.g., sunburn), or the use of certain types of drugs (e.g., steroids, calcium channel blockers, thiazolidinediones, non-steroidal anti-inflammatories (NSAIDs), and estrogens).

One method of detecting the presence of edema is to determine fluid volume change in the patient. Different technologies have been developed to identify volume change, including those based on water displacement, weight changes, limb measurements, light-emitting diode (LED) volume scanning and duel X-ray absorptiometry. However, these methods can be difficult to use, inaccurate, and/or can be expensive and require specialized equipment.

The most widely used cflinical method for assessing edema is digital manipulation, also known as the “pitting” method. This assessment is accomplished by pressing into the patient's skin (e.g., on the patient's leg) near a bony surface (e.g., the tibia) and qualitatively evaluating the degree of pitting. Pitting is the indentation in the swollen tissue that remains following removal of the pressure from the edematous area. Due to the altered tissue composition resulting from edema, there can be a putty-like consistency to the tissue, and the tissue can remain in the indented position for several seconds or minutes before returning to its original form. The individual (e.g., the doctor or other health care provider) performing the test assesses one or more of the depth of the indentation, how much force is required to reach a nearby bone, how long the tissue takes to return to its original state, and skin quality. The level of edema is typically described using a ranking system of one to four (slight to severe).

Because peripheral edema can be a physical manifestation of a number of different disease conditions, there is an ongoing need for additional methods for measuring edema that are more accurate and/or less subjective. In particular, there is an ongoing need for additional methods of measuring edema that are reliable and economical; fast and easy to use; that provide repeatable measurements (from day to day and/or from practitioner to practitioner); and that have the potential to be used at home, as well as in conventional and out-patient health-care and veterinary settings.

### SUMMARY

In some embodiments, the presently disclosed subject matter provides a device for measuring edema and/or one or more skin-related properties, optionally wherein the one or more skin-related properties are selected from elasticity, thickness, quality, age, or hydration level, said device comprising: (a) a nozzle configured for directing at least one pulse of compressed air or other gas from a first end of said nozzle to a first portion of a skin surface of a subject; and (b) one or more camera configured to record one or more images of the first portion of the skin surface. In some embodiments, the device further comprises a housing, wherein said housing comprises a first opening for the first end of the nozzle and at least a second opening for one or more lens of the one or more camera, optionally wherein said housing comprises a thermoplastic or thermosetting polymer.

In some embodiments, the housing further comprises a positioning arm, wherein said positioning arm extends from a main body of the housing and wherein, when an end of said positioning arm is placed directly adjacent to a second portion of the skin surface of the subject, said positioning arm controls one or more of the group consisting of a first distance at which the first end of the nozzle is positioned relative to the first portion of the skin surface of the subject; a first angle at which the first end of the nozzle is positioned relative to the first portion of the skin surface of the subject; a second distance or distances at which one or more lens of the at least one camera is positioned relative to the first portion of the skin surface of the subject; and a second angle or angles at which the one or more lens of the at least one camera is positioned relative to the first portion of the skin surface of the subject. In some embodiments, the housing comprises a handle or hand grip and/or the device is configured to comprise at least one hand-held component. In some embodiments, the housing comprises a triggering mechanism configured to initiate one or both of the release of one or more pulse of compressed air or other gas from the first end of the nozzle and the recording of one or more images by the at least one camera.

In some embodiments, the device comprises a signal processing unit configured to analyze the one or more images and calculate one or more measurements related to an indentation in the first portion of the skin surface caused by contact of said first portion of the skin surface with the at least one or more pulses of compressed air or other gas, optionally wherein each of said one or more measurements is selected from the group comprising a rate of indentation, a depth of indentation, an indentation area, a change in indentation area as a function of time, and a measurement of one or more topological features of the indentation, further optionally wherein the signal processing unit comprises a microprocessor present in a housing of said device. In some embodiments, the signal processing unit is configured to analyze a plurality of images recorded sequentially over a pre-determined period of time after said first portion of the skin surface is contacted with the one or more pulses of compressed air or other gas and calculate the change in indentation area in the first portion of the skin surface as a function of time. In some embodiments, the signal processing unit is further configured to calculate an edema score from the one or more measurements, optionally wherein the one or more measurements comprise a change in indentation area as a function of time, further optionally wherein the edema score is a rating of severity of edema on a numerical scale, such as a scale of 1.00 to 4.00.

In some embodiments, the device further comprises a display window for displaying one or more measurements related to an indentation and/or an edema score. In some embodiments, the device further comprises a data storage unit for storing one or more measurements related to an indentation and/or an edema score obtained at different times of the same day, week or month; using different portions of the skin surface of the subject; and/or obtained using different subjects.

In some embodiments, the device further comprises a source of compressed air or other gas attached to a second end of the nozzle, optionally wherein the source of compressed air or other gas is selected from one or more of the group consisting of a table top air compressor, a compressed gas canister or cartridge, and a gas tank. In some embodiments, a second end of the nozzle is attached to a hose or tubing that is detachably connected to a compressed air or other compressed gas source internal to or external to a housing of said device.

In some embodiments, the device further comprises a gas pressure monitor and/or a device for regulating gas pressure of the pulse of compressed air or other compressed gas exiting the first end of the nozzle. In some embodiments, the device further comprises one or more batteries configured to provide power to the device, optionally wherein the one or more batteries are rechargeable batteries. In some embodiments, the device further comprises a component configured to wirelessly communicate data from the one or more camera to one or more of a portable computing device, a desktop computing device, a server computer, a persistent storage device, and/or a network. In some embodiments, the device further comprises one or more light source configured to provide light to the first portion of the skin surface of the subject, optionally wherein said one or more light source comprises a light emitting diode (LED).

In some embodiments, the device comprises a positioning arm comprising one or more outlets for the first end of the nozzle, at least one lens of the one or more camera, and light from the one or more light source, optionally wherein the positioning arm has a curved surface and/or wherein the one or more outlets are recessed within the positioning arm to protect the one or more outlets from ambient light. In some embodiments, the device is portable. In some embodiments, the device is configured to be attachable to an assistive device, optionally wherein the assistive device is selected from the group consisting of a cane, a walker, a wheelchair, and a crutch.

In some embodiments, the presently disclosed subject matter provides a method of measuring edema in a subject, the method comprising: (a) directing one or more pulses of compressed air or other gas at a portion of a skin surface of the subject, optionally wherein the portion of the skin surface is a skin surface of an arm, hand, wrist, face, leg, foot, or ankle; (b) obtaining one or more images of said portion of the skin surface; (c) analyzing the one or more images of said portion of the skin surface to calculate an area of an indentation resulting from contact of the skin surface with the one or more pulses of compressed air or other gas; and (d) determining a measure of edema severity in the subject using the area of indentation, optionally wherein the measure of edema severity is provided as a value on a numerical scale, further optionally wherein the numerical scale is between 1.00 and 4.00.

In some embodiments, each of the one or more pulses of compressed air or other gas has a pressure of between about 1 and about 100 psi, optionally wherein the pressure is about 50 psi. In some embodiments, each of the one or more pulses of compressed air or other gas has a duration of between about 0.1 second and about 30 seconds, optionally wherein the duration is between about 0.2 seconds and about 10 seconds. In some embodiments, the one or more pulses of compressed air or other gas is a series of pulses, optionally wherein the series of pulses comprises pulses of different pressures, further optionally wherein the series of pulses is directed at the portion of the skin surface of the subject in order from the highest pressure pulse to the lowest pressure pulse or from lowest pressure pulse to highest pressure pulse, thereby providing a pulsed pressure gradient.

In some embodiments, obtaining one or more images comprises obtaining a plurality of images of the skin surface at a series of sequential time points after the skin surface is contacted by the one or more pulses of compressed air or other gas, and wherein analyzing the one or more images comprises analyzing the plurality of images of the skin surface to calculate indentation area as a function of time and/or the maximum depth of indentation. In some embodiments, the plurality of images comprises at least about 10 images, optionally wherein the plurality of images is obtained over a period of time from about 0.1 second to about 5 minutes.

In some embodiments, the indentation area is calculated as a pixel count of a binarized image of the portion of the skin surface indented by the one or more pulse of compressed air or other gas. In some embodiments, the steps of directing one or more pulses of compressed air or other gas, obtaining one or more images, analyzing one or more images, and determining a measure of edema severity are repeated after one or more minutes, hours, days, or weeks to determine progression or regression of edema in the subject over time.

In some embodiments, the subject is a mammal, optionally wherein the mammal is a human, and/or wherein the subject is pregnant, suffering from a burn or a known or suspected allergic reaction, is being treated for a medical condition with a drug or procedure that can result in peripheral edema, or has been diagnosed with, is suspected of having, or is at risk of developing congestive heart failure (CHF), Imphedema, liver disease, kidney disease, or venous stasis disease. In some embodiments, the subject has a disease associated with peripheral edema, optionally wherein the disease is congestive heart failure (CHF), and wherein measuring peripheral edema in the subject is performed as part of monitoring the severity of the disease, optionally wherein one or more aspect of the treatment of the subject for the disease is adjusted based on the measure of edema. In some embodiments, the subject has been diagnosed with or is at risk of lymphedema, optionally wherein the subject is a cancer patient, further optionally wherein the subject is a cancer patient who has had one or more lymph nodes surgically removed and/or who has received radiotherapy.

In some embodiments, the method is performed using a device comprising: (a) a nozzle configured for directing at least one pulse of compressed air or other gas from a first end of said nozzle to a first portion of a skin surface of a subject; and (b) at least one camera configured for recording one or more images of the first portion of the skin surface, optionally wherein the one or more images comprise optical or digital images. In some embodiments, the first end of said nozzle is between about 0.2 inches and about 1.5 inches from the skin surface of the subject, optionally wherein said first end of said nozzle is about 1 inch from the skin surface of the subject. In some embodiments, the one or more pulses of air are directed to the skin surface of the subject at an angle of incidence between about 45 degrees and about 75 degrees, optionally of about 60 degrees.

In some embodiments, the method further comprises illuminating the skin surface of the subject with light, optionally light from a LED light source. In some embodiments, the light and the one or more pulses of compressed air or other gas are directed toward the skin surface of the subject at an angle of incidence of about 0 degrees, and wherein the camera is configured to capture light reflected from the skin surface of the subject at an angle of about 45 degrees. In some embodiments, the illuminating is performed in combination with protecting the skin surface of the subject from ambient light.

In some embodiments, the presently disclosed subject matter provides a system for measuring and/or monitoring edema and/or one or more skin-related properties in a subject, the system comprising: (a) a device for measuring edema and/or one or more skin-related properties, said device comprising: (i) a nozzle configured for directing at least one pulse of compressed air or other gas from a first end of said nozzle to a first portion of a skin surface of a subject; (ii) one or more camera configured for recording one or more images of the first portion of the skin surface; and (iii) a communications component configured to transmit data, optionally wherein the data is selected from optical and/or digital images collected by the one or more camera; and (b) at least one of a remote server computer and/or a personal computing device configured to receive data from the device for measuring edema and/or one or more skin-related properties.

Accordingly, it is an object of the presently disclosed subject matter to provide devices, systems, and methods to measure and/or monitor edema and/or skin properties.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIG. 1 is a schematic drawing showing an exemplary device for measuring edema according to an embodiment of the presently disclosed subject matter.

FIG. 2A is a schematic diagram of a method of analyzing images of a skin indentation according to an embodiment of a method of the presently disclosed subject matter.

FIG. 2B is a line drawing of a photograph of an indentation in an artificial edematous skin sample.

FIG. 2C is a graph showing an example of indentation area (in pixels) versus time (frame number) data collected according to an embodiment of the presently disclosed method.

FIG. 3 is a side view of an exemplary device for the preliminary assessment of measuring edema according to a method of the presently disclosed subject matter.

FIG. 4 is a pair of images of air puff/pulse indentation in an artificial skin sample. The air incidence angle was 60 degrees (°) and the camera was positioned directly above the skin sample. The image on the left is a cropped and magnified camera image of indented artificial skin prior to binarization processing, and the image on the right is the image after processing.

FIG. 5 is a graph showing the indentation area, in pixels, from a puff of compressed air over time (in milliseconds (ms)) for 10 separate trials.

FIG. 6A is a graph showing indentation area (in pixels) versus time (in milliseconds (ms)) in skin samples representing four levels of edema (1+, 2+, 3+, and 4+).

FIG. 6B is a graph showing the mean and standard deviation of the indentation area under the curve (AUC), in pixels, for skin samples representing four levels of edema (1+, 2+, 3+, and 4+).

FIG. 6C is a graph showing the mean and standard deviation of the maximum indentation area (MIA), in pixels, for skin samples representing four levels of edema (1+, 2+, 3+, and 4+).

FIG. 7 is a pair of images of air puff skin indentation taken of an indented skin sample with dark skin tone. The image on the left is the magnified and cropped camera image prior to binarization and threshold segmentation processing, and the image on the right is after processing.

FIG. 8 is a pair of images taken of a skin sample with dark hair. The image on the left is the magnified and cropped camera image prior to binarization and baseline threshold segmentation processing, and the image on the right is after processing. Individual hairs are seen as long rods that can be tagged and excluded from an indentation area measurement.

FIG. 9A is a front perspective view of an exemplary hand-held device for measuring edema according to an embodiment of the presently disclosed subject matter.

FIG. 9B is a cutaway side view of the device shown in FIG. 9A.

FIG. 9C is a rear view of a display window of the device shown in FIG. 9A.

FIG. 10 is a schematic drawing showing exemplary nozzle, light, and camera placement relative to a skin surface according to an embodiment of the presently disclosed subject matter.

FIG. 11A is a front perspective view of an exemplary device for measuring edema according to an embodiment of the presently disclosed subject matter.

FIG. 11B is a front perspective view of the device shown in FIG. 11A in which a housing of the device is shown as transparent to show the arrangement of elements therein.

FIG. 11C is a top view of a portion of a variation of the device shown in FIGS. 11A and 11B.

FIG. 11D is a bottom view of a portion of a variation of the device shown in FIGS. 11A and 11B.

FIG. 12 is a schematic drawing showing an exemplary gas delivery line that can regulate compressed air or other gas to standardize air/gas pulses.

FIG. 13A is a side perspective view of an exemplary device according to an embodiment of the presently disclosed subject matter that can be attached to an assistive component.

FIG. 13B is a front view of the device of FIG. 13A.

FIG. 13C is a top view of the device of FIG. 13A shown with its protective cover open.

FIG. 14 is a front view of an exemplary two-part portable device of the presently disclosed subject matter.

FIG. 15 is a schematic drawing of an exemplary system for measuring edema according to an embodiment of the presently disclosed subject matter.

### DETAILED DESCRIPTION

The presently disclosed subject matter will now be described more fully hereinafter with reference to the accompanying Examples and Figures, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

All publications, patent applications, patents, and other references mentioned herein are incorporated by reference in their entirety.

### I. Definitions

While the following terms are believed to be well understood by one of ordinary skill in the art, the following definitions are set forth to facilitate explanation of the presently disclosed subject matter.

Unless otherwise defined, all technical and scientific terms used herein have the same meaning as commonly understood by one of ordinary skill in the art to which this presently described subject matter belongs.

Following long-standing patent law convention, the terms “a”, “an”, and “the” refer to “one or more” when used in this application, including the claims. Thus, for example, reference to “a pulse” or “a camera” includes a plurality of such pulses or cameras, and so forth.

Unless otherwise indicated, all numbers expressing quantities of time, pressure, distance, angle degree, weight, height, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a weight, temperature, pressure, angle, distance, depth, area, volume, percentage, time, rate, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed devices or systems.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “edema” as used herein refers to the abnormal accumulation of fluid in cavities or tissues in the body (e.g., the lungs, brain, abdomen, chest or extremities) resulting in swelling. “Peripheral edema” refers to edema in tissues or cavities in the extremities (e.g., the feet, ankles, legs (e.g., calves), arms, wrists, and/or hands) that are perfused by the peripheral vascular system.

“Lymphedema” is a form of edema, usually peripheral edema, caused by blockage or other damage in the lymph system.

The terms “compressed air or other gas” and “compressed air or other compressed gas” as used herein can refer to any compressed gas or compressed gas mixture. In some embodiments, the compressed gas can comprise a gas or mixture of gases having, in a container, an absolute pressure of greater than about 40 psi at about room temperature (i.e., about 21.1° C.). In some embodiments, the compressed gas is a non-toxic, non-corrosive, and or non-flammable compressed gas. In some embodiments, the compressed gas comprises compressed air; compressed nitrogen; compressed carbon dioxide; compressed nitrous oxide; compressed helium; compressed argon, compressed neon or another compressed inert gas; or any mixture thereof. In some embodiments, the compressed gas is a mixture comprising at least two gases selected from nitrogen, oxygen, argon, neon, helium, and carbon dioxide. However, the gas of the presently disclosed subject matter is not limited to such gases and can include any molecule or mixture of molecules that can be present in gaseous form. The mixture can be compressed air (e.g., compressed dry air, which can comprise, by volume, about 78% nitrogen, about 21% oxygen, about 1% argon, and less than 1% of each of neon, carbon dioxide, helium, and methane) or can contain two or more of the same gases (nitrogen, oxygen, argon, carbon dioxide, neon, helium, and methane) present in compressed air but at a volume % or relative volume % that is different from that of compressed dry air. The compressed gas can be provided in any suitable container, e.g., a tank, canister or cartridge. Compressed gas can also be provided from a “house line” or “in house line”, i.e., an outlet from compressed gas system present in a building such as a hospital, laboratory, medical or veterinary clinic, or doctor's office building. The compressed gas (e.g., compressed air) can also be provided using a compressor e.g. a wall, table-top, and/or portable compressor unit, or using a pump (e.g., a manual pump).

The term “nozzle” as used herein refers to a pipe or tube used to direct or modify the flow of a fluid (e.g., a liquid or gas). For example, the nozzle can be used to control the rate of flow, speed, direction, shape and/or pressure of a stream or pulse of gas (e.g., compressed air). More particularly, in some embodiments, a nozzle can be used to control the direction or other characteristics of a gas flow from an enclosed chamber or pipe. In some embodiments, the nozzle is a metal or plastic pipe or tube having a cylindrical, conical, or round spout at one end.

The terms “pulse” and “puff” as used herein can refer to a short burst of a fluid (e.g., a gas). A pulse can last for less than one second (e.g., between about 0.1 and about 0.9 seconds) to up to a few minutes (e.g., about 1 minute or about 2 minutes).

### II. General Considerations

Edema is often assessed by medical professionals to gain insights related to a variety of diagnosed and potential medical conditions. For example, edema (e.g., peripheral edema) can be related to injuries, such as burns; allergic reactions; too much dietary salt intake, pregnancy, and with the use of some medications (e.g., steroids, calcium channel blockers, thiazolidinediones, NSAIDS, and estrogens). Peripheral edema is also associated with chronic diseases and disorders, such as congestive heart failure (CHF), liver disease (e.g., cirrhosis), kidney disease, lymphedema (LE), hypoalbumenia, and chronic venous insufficiency.

According to the conventional “pitting” method of measuring edema, a health-care provider presses on the skin of a patient with his or her finger and provides a score for the patient's edema according to a relative scale, typically from 1+ (slight) to 4+ (severe). The score is assigned based on one or more criteria including the depth of the pitting, the general appearance of the extremities (e.g., the feet, ankles, hands, etc.), the amount of time required for the pit to disappear, and the amount of force required to form the pit. Table 1 below shows the criteria typically used to assess pitting edema. The recovery times associated with each particular edema score described in Table 1 are exemplary, and can vary from provider to provider. In addition, criteria such as force required for pitting and general appearance are subjective. Thus, the edema score provided by the conventional “pitting” method can vary based upon the individual measuring the edema. The same individual can also measure edema inconsistently from day to day due to the subjective nature of the measurements.

TABLE 1
Assessment of Pitting Edema.

| | Edema Score | | | |
|---|---|---|---|---|
| Criteria | 1+ | 2+ | 3+ | 4+ |
| Depth of | 2 mm or less | 2-4 mm | 4-6 mm | 6-8 mm |
| pitting | | | | |
| Recovery | Pitting | Disappears in | Can last more | Lasts as long |
| time | disappears | 10-15 | than 1 minute | as 2-5 |
| | rapidly | seconds | | minutes |
| Visual | No visible | No readily | Dependent | Dependent |
| appearance | distortion | detectable | extremity | extremity is |
| | | distortion | looks fuller | grossly |
| | | | and swollen | distorted |

Lymphedema (LE) is characterized by excess protein and fluid in the tissue. Primary LE is caused by abnormal development of the lymph system. Secondary LE, which accounts for about 99% of LE, is generally caused by damage from cancer, removal of lymph nodes, radiation, chemotherapy, surgery, an immune disorder, or an infection (e.g., as in the case of filariasis, which is caused by a parasitic worm of the species Wuchereria bancrofti). In particular, secondary LE, which can cause dramatic limb swelling as well as pain and heaviness in the affected limb or limbs, is a significant survivor issue for cancer patients (e.g., breast cancer and head and neck cancer patients), resulting in poorer physical and mental quality of life due to pain, physical and functional limitations, psychological consequences such as depression and anxiety, and increased economic burdens. Over time, LE can result in hardening of the tissues. Further, if untreated, LE can result in infections (i.e., cellulitis) and, eventually, ulcerations.

There is currently no single method that can assess LE across all stages. The progression of LE is classified by stages (0-3) as defined by the International Society of Lymphology. In stage 0 (latent or subclinical), fluid accumulation is not always visibly evident despite impairment of lymph flow and subtle changes in tissue composition and symptoms. In stage 1, there is visible swelling (pitting edema), however, the swelling is reversible. With Stage 2 LE, the tissues become fibrotic (hardened); and in the final stage, Stage 3, the tissues become even harder, and the damage is not reversible. Thus, recognizing LE early and treating it properly is the best way to manage the condition. Like the pitting method of staging edema, the staging of LE is determined by a health care provider based on physical examination of the affected limb; an approach prone to observer bias and in which early detection can be challenging. Further, the above-described staging method only involves the physical condition of the affected region; and, therefore, does not consider functional changes or subtle changes in tissue constituents. By being able to more accurately and easily measure edema (and/or other skin properties) in subjects at risk of lymphedema, treatment can be provided while the disease is still treatable.

The pitting edema test described hereinabove mimics indentation tests that are used to determine material properties. In these tests, the force application can be approximated as a step force that is held constant for a short duration and then removed. Most biological materials are viscoelastic and the typical displacement response involves creep (increased displacement with constant force) and then relaxation when the force is removed. The creep response reflects the properties of the skin and the subcutaneous soft tissue. Thus, it can provide insights regarding possible protein buildup (e.g., in lymphedema). The relaxation process, particularly the recovery time, can be related to the fluid volume accumulated underneath the skin.

Accordingly, the presently disclosed subject matter is based in part on methods of modeling the behavior of viscoelastic materials using lumped parameter models such as, but not limited to, the Standard Linear Solid (SLS) model, the Kelvin-Voight model, poroelastic models and poroviscoelastic models. Within these models, the elastic (instantaneous) response of soft tissues can be modeled with a linear spring (E) and the viscous nature can be modeled with a damper or dashpot (η). Applying these models to edematous tissue, the viscous and elastic characteristics of the involved springs (Es) and damper (η), e.g., in either the SLS model or the Kelvin-Voight model can be determined. Taking the SLS model as an example, the compliance equation for the incident creep and the end relaxation equation are presented by Equations (1) and (2), below, respectively.

$$\begin{matrix}{I}_{o} ( t ) = \frac{{E}_{1} + {E}_{2} - {E}_{1} {e}^{\frac{- {E}_{2} {E}_{1} t}{{η}_{1 ( {E}_{2} + {E}_{1} )}}}}{{E}_{2} ( {E}_{1} + {E}_{2} )} & ( 1 ) \\ {R}_{0} ( t ) = {E}_{2} + {E}_{1} {e}^{\frac{- {E}_{1} t}{{η}_{1}} + {E}_{2}} & ( 2 )\end{matrix}$$

where I₀(t) and R₀(t) are related to strain and stress, respectively, determined experimentally. Determination of these properties from the presently disclosed methods and/or measurements from the presently disclosed devices can be used in the classification of edema and lymphedema, as well as other skin properties (e.g., elasticity, hydration level, etc.).

Building on this framework, it can be noted that equations (1) and (2) above are based on stress and strain and are independent of geometry. The presently disclosed methods can collect data related to indentation area as a function of time and a known pressure applied as a step. Assuming that indentation area is a surrogate for displacement and the force F can be approximated from the known pressure, tissue displacement can be solved as a function of the tissue properties as seen in Equation (3):

$$x = \frac{F}{{k}_{2}} ( 1 - ( \frac{{k}_{1}}{{k}_{1} + {k}_{2}} ) {e}^{\frac{- t}{τ}} , \qquad ( 3 )$$

$$where τ = \frac{{η}_{1}}{{k}_{1}} ( 1 + \frac{{k}_{2}}{{k}_{1}} ) ,$$

where x is displacement, k₁ and k₂ represent tissue stiffness, and τ is the time constant. Curve fitting methods can be used to fit Equation 3 to an experimental indentation area curve. Since these parameters are not independent of the geometry, the impact of normalizing area by limb circumference or BMI can be assessed and corrected for, as necessary.

In some embodiments, the presently disclosed subject matter provides devices and methods for the fast and accurate measurement of edema (e.g., peripheral edema, including LE) in a user-independent manner. Thus, the presently disclosed devices and methods can be used to provide for improved comparability of edema scores taken at different times and/or measured by different individuals (e.g., doctors, veterinarians, nurses, nursing assistants, etc.). However, the presently disclosed devices and methods can also be used to assess various skin properties, as well, such as, but not limited to, elasticity, thickness, hydration level, skin quality (e.g., quality relative to skin of a typical individual), and/or skin “age” (e.g., skin quality relative to the skin quality of a typical individual of the same biological age). Thus, for example, the methods and/or devices can be adapted to provide a more quantitative measure of skin health and/or skin damage resulting from, for instance, sun or wind exposure. Further, while in some embodiments, the devices and methods are used to measure peripheral edema and/or a skin property using a portion of the skin surface of an extremity, such as an arm or leg, the devices and methods can be adapted for use to measure edema and/or a skin property of any skin surface, such as the face, neck, chest, back, abdomen, etc. Thus, in some embodiments, the method can be adapted for use in dermatology, e.g., with regard to the assessment of various skin disorders or conditions, or in the cosmetics or personal care industry, e.g., to measure the skin effects of cosmetic and/or skin care products.

### III. Methods

In some embodiments, the presently disclosed subject matter provides a method of measuring edema (e.g., peripheral edema) in a subject. In some embodiments, the method comprises: (a) directing one or more pulses of compressed air or other compressed gas (e.g., carbon dioxide, nitrogen, or mixtures thereof) at a portion of a skin surface of the subject; (b) obtaining one or more images of said portion of the skin surface; and (c) analyzing the one or more images of said portion of the skin surface, e.g., to measure one or more aspect of the indentation made in the skin surface by the one or more pulses of compressed air or other compressed gas. Such aspects can include, but are not limited to, the area of indentation (e.g., at a particular time following indentation with a pulse of a particular pressure and/or duration), the depth of indentation, the rate at which the indentation disappears, the maximum depth or area of indentation produced by the pulse or pulses, the length of time or the amount of pressure needed for the indentation to reach a particular depth or area, the amount of time for the indentation depth to fully recover or to recover to a particular percentage of the maximum indentation depth after one or more pulse of compressed air or other gas (e.g., the “recovery time”), the difference in area or depth of indentation produced by pulses of different pressure, and the indentation area as a function of time. In some embodiments, step (c) comprises analyzing the one or more images of said portion of the skin surface to calculate the area of an indentation resulting from contact of the skin surface with the one or more pulses of compressed air or other gas.

In some embodiments, the method can further comprise step (d): determining a measure of edema severity (i.e., an “edema score”) in the subject using the area of the indentation calculated in step (c) (or another measurement determined from step (c)). In some embodiments, the measure of edema severity is provided as a value on a numerical scale. For example, in some embodiments, the measure of edema severity is correlated to the conventional scale used in “pitting” edema measured by pressing a finger in an affected area. Thus, in some embodiments, the numerical scale is between 1.00 and 4.00, e.g., wherein a higher number corresponds to higher severity edema.

In some embodiments, the portion of the skin surface is a skin surface of an arm (e.g., forearm), wrist, hand, face, leg (e.g., calf), foot, or ankle. However, the skin surface can be any other skin surface, e.g., the chest, back, or abdomen. In some embodiments, the skin surface can include portions of skin surface from two different locations (e.g., the ankle and the calf, the left ankle and the right ankle, two sides or locations of the same ankle or leg, the ankle and the arm, etc.).

The pressure of the one or more pulses of compressed air can be varied depending upon the subject, edema cause, and/or skin surface being assessed. For example, lower pressure pulses can be used for older subjects with thinner and/or easier to bruise skin or for subjects suffering from edema related to a burn. Lower pressure pulses can also be used on skin surfaces that tend to be more sensitive, e.g., the inner wrist or face, or where the skin surface is closer to the bone. In some embodiments, a pulse pressure can be selected to provide an indentation of a predetermined minimum depth, e.g., 2 mm. In some embodiments, each of the one or more pulses of compressed air or other gas has a pressure of between about 1 and about 100 psi (e.g., about 1, 5, 10, 15, 20, 25, 30, 35, 40, 45, 50, 55, 60, 65, 70, 75, 80, 85, 90, 95 or about 100 psi). In some embodiments, such as when the subject has more advanced/later stage LE where tissues have started to become more fibrotic and/or stiffer, a pulse pressure above 100 psi can be used. In some embodiments, the pressure is about 50 psi. In some embodiments, the pressure is about 40 psi.

The duration of the pulse or pulses can also be varied. For example, the duration of the pulse can be selected based the minimum duration needed to provide an indentation that remains long enough to be imaged (e.g., by a high-speed camera) and/or to provide an indentation of a predetermined minimum depth or area. In some embodiments, increased duration of the pulse can allow for the observation of creep. In some embodiments, the duration of the pulse is selected to correspond to the maximum duration that does not cause significant pain or discomfort to a subject. In some embodiments, the duration of the pulse is selected to correspond to the maximum amount of time that a nozzle and/or camera can be held still in order to provide the pulse and/or record an image of the resulting indentation. In some embodiments, each pulse has a duration of between about 0.1 second and about 30 seconds (e.g., about 0.1, 0.2, 0.3, 0.4, 0.5, 0.6, 0.7, 0.8, 0.9, 1, 2, 3, 4, 5, 10, 15, 20, 25, or about 30 seconds). In some embodiments, each pulse has a duration between about 0.2 seconds and about 10 seconds.

In some embodiments, the one or more pulses of compressed air or other gas is a series of pulses (i.e., at least two, three, four, five, six, seven, eight, nine, or ten pulses or more). In some embodiments, the series of pulses comprises individual pulses of different pressures. In some embodiments, the series of pulses is directed at the portion of the skin surface in increasing or decreasing order based on pressure (i.e., from lowest to highest pressure or from highest to lowest pressure), thereby providing a pulsed pressure gradient. In some embodiments, the pulsing can act as a vibration, e.g., to provide additional information about the tissue/skin.

In some embodiments, step (b) comprises obtaining a plurality of images of the skin surface at a series of sequential time points after the skin surface is contacted by the one or more pulses of compressed air or other gas. In some embodiments, step (c) comprises analyzing a plurality of images of the skin surface taken sequentially over time to calculate indentation area as a function of time. The number of images to be captured and the rate at which the images are captured can be selected to collect sufficient data to show incremental change in the skin surface in response to the one or more pulses. In some embodiments, the plurality of images comprises at least about 10, 25, 50, 75, 100, 150, 200, 250, 500, or about 1,000 images. In some embodiments, the plurality of images comprises between about 50 and about 300 images. In some embodiments, the plurality of images is obtained over a period of time from about 0.1 second to about 5 minutes (e.g., about 1 second, 30 seconds, 60 seconds, 2 minutes, 3 minutes, 4 minutes, or about 5 minutes, starting for example, at the time the first pulse of compressed air or other gas impinges the skin surface). In some embodiments, the plurality of images is obtained over a longer period of time, e.g., up to about 10 minutes or up to about 15 minutes.

In some embodiments, the one or more images comprise one or more optical and/or digital images, e.g., obtained using a video camera, a high-speed camera, and/or a stereo camera. In some embodiments, the indentation area is calculated as a pixel count of a binarized image or images of the portion of the skin surface indented by the one or more pulse of compressed air or other gas. In some embodiments, step (c) comprises: cropping one or more optical or digital images of a skin area indented with one or more pulse of compressed air or other gas (e.g., to center the indented area in the image, to substantially exclude skin surface outside the area of the indentation, and/or to provide an image wherein the indented skin surface comprises greater than about 50, 55, 60, 65, 70, 75, 80, 85, 90, 95% or more of the image); binarizing the image (e.g., rendering the image in black and white, optionally based on a particular threshold greyscale level, such as that determined from initial images of the skin surface); and calculating the dent area (e.g., by counting the number of black pixels in the image). In some embodiments, the dent area is plotted over time. In some embodiments, the pixel count is corrected or normalized based on a pixel count from one or more images of the same skin surface prior to indentation or within the first few milliseconds of impingement with a pulse of compressed air or other gas.

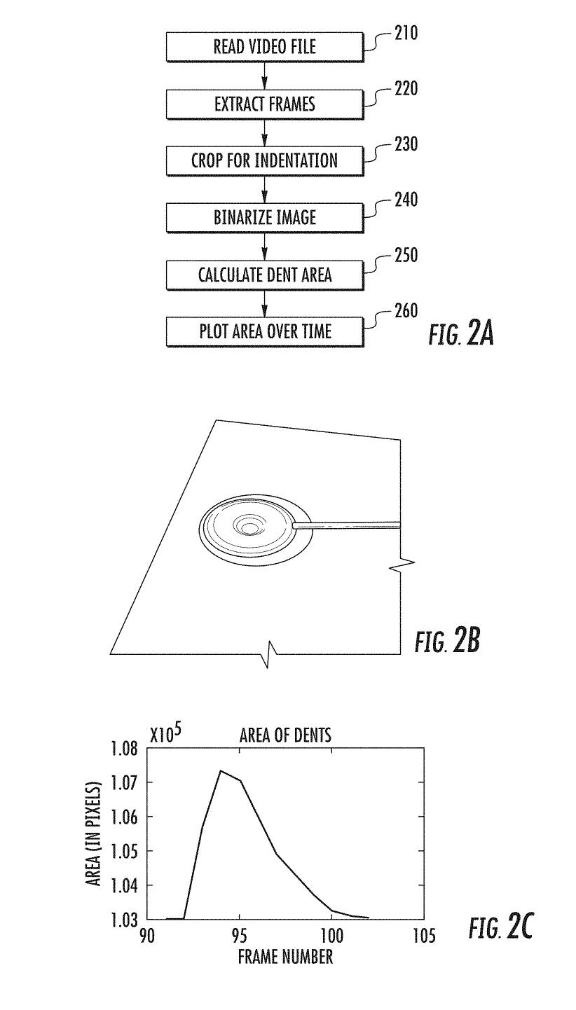

With more particular regard to the presently disclosed methods, FIG. 2A shows a flow chart according to an embodiment of a method for analyzing images obtained of a skin surface during and/or after indentation. As shown in FIG. 2A, the analysis can comprise step 210 in which a video file is read. For example, the video file can be obtained from a video camera taking a video of a skin surface for a period of time immediately following indentation resulting from contact with one or more pulses of compressed air or other gas. The method further comprises step 220 in which individual image frames (e.g., between about 10 and about 1,000 frames, between about 50 and about 500 frames or between about 150 and about 250 frames) are extracted from the video file (i.e., of particular time points/frame numbers following indentation). In step 230, the individual extracted images are cropped as desired to remove non-indented regions of the skin surface. In some embodiments, the method includes step 240 in which each image is binarized to provide an image that is black and white only. In some embodiments, the method can further comprise a step 250 comprising calculating the indentation (i.e., “dent”) area in each frame and a step 260 comprising plotting the indentation area over time.

FIG. 2B shows a drawing of an uncropped image of an artificial skin sample indented using a pulse of compressed air. FIG. 2C shows a graph of dent area over time for the artificial skin sample, wherein time progression is indicated by increasing frame number. Indentation area is measured as a function of pixel number from the binary images.

In some embodiments, an edema score can be calculated by determining a percent difference in area under the curve in a plot of indentation area over time obtained from the subject as compared to a plot obtained from a comparable skin surface in a subject who is free of edema.

Initial testing of the presently disclosed methods was performed using an artificial skin sample instilled with water as a model for edema and a non-water instilled sample as a model of the absence of edema. The artificial skin samples were impinged with a 50 psi pulse of compressed air and images of the indentation area were obtained with a high speed camera capable of taking about 30 frames per second. Plots of indentation area (in pixels) versus time (in frame number) were prepared based on data extracted from the images. Generally, plots of area of indentation versus time for the non-edema model showed a steep increase in area of indentation followed by a relatively quick recovery, providing relatively sharp peaks, while the plots for the edema model provided broader peaks.

In some embodiments, steps (a)-(c) (i.e., directing one or more pulses of compressed air or other gas at a portion of a skin surface of a subject; obtaining one or more images, and analyzing one or more images) or steps (a)-(d) (i.e., directing one or more pulses of compressed air or other gas at a portion of a skin surface of a subject; obtaining one or more images, analyzing one or more images, and determining a measure of edema severity) of the presently disclosed methods are repeated after one or more minutes, hours, days, or weeks to determine potential progression or regression of edema in the subject over time. Typically, the subject can be a mammal, such as a human, or a mammal of importance due to being endangered (such as a Siberian tiger), of economical importance (an animal raised on farms for consumption by humans) and/or social importance (an animal kept as a pet or in a zoo) to humans, for instance, a carnivore other than a human (such as a cat or dog), a swine (a pig, hog, or wild boar), a ruminant (such as cattle, oxen, sheep, giraffes, deer, goats, bison, and camels), and a horse (e.g., a race horse). In some embodiments, the subject is a bird (e.g., a parrot). In some embodiments, the subject is a human.

In some embodiments, the subject is a subject who is suspected of having, who is at risk of developing, or who has been diagnosed with a disease or condition associated with edema (e.g., peripheral edema). In some embodiments, the subject is pregnant, suffering from a burn or a known or suspected allergic reaction, is being treated for a medical condition with a drug or using a medical procedure (e.g., radiation or surgery) that can result in edema, or who has been diagnosed with, is at risk of developing, or is suspected of having a condition selected from, but not limited to, congestive heart failure (CHF), lymphedema, liver disease, kidney disease, cancer, and venous stasis disease. In some embodiments, the subject has a disease associated with peripheral edema (e.g., CHF), and measuring peripheral edema in the subject is performed as part of monitoring the severity of the disease. In some embodiments, one or more aspect of the treatment of the subject for the disease (e.g., medication type or dose) or for the associated edema is adjusted based on the measure of edema. Conventional treatment for edema typically includes one or more of: the administration of a diuretic (e.g., furosemide), exercise of muscles in the affected body area, the use of compression garments or devices (e.g., pneumatic compression devices), massage, and following a reduced sodium diet. In some embodiments, one or more of these treatment options can be added, deleted, or otherwise adjusted to a subject's overall treatment regimen based on the measure of edema.

In some embodiments, the subject is suspected of having, is at risk of developing, or has been diagnosed with lymphedema. In some embodiments, the subject has been diagnosed with or is suspected of having primary lymphedema. In some embodiments, the subject has been diagnosed with, is suspected of having, or is at risk of developing secondary lymphedema. For example, in some embodiments, the subject has had one or more lymph nodes removed. In some embodiments, the subject has been treated with or otherwise exposed (e.g., accidentally exposed) to a chemotherapeutic agent and/or radiation. In some embodiments, the subject is a subject who has been treated for cancer (e.g., with surgical lymph node removal and/or radiation). In some embodiments, the cancer is a breast cancer or a head and neck cancer. In some embodiments, the subject is a subject who has been treated for breast cancer and the lymphedema is breast-cancer related lymphedema (BCRL). In some embodiments, the subject is a breast cancer patient who has undergone lumpectomy and/or mastectomy and/or who has undergone radiation treatment (e.g., to the chest and/or upper arm area). In some embodiments, the subject is a breast cancer patient who has undergone a modified radical mastectomy.

In some embodiments, the presently disclosed method is performed using a device of the presently disclosed subject matter, such as a device as described further hereinbelow. Thus, for instance, in some embodiments, the method is performed using a device comprising: (a) a nozzle configured for directing at least one pulse of compressed air or other gas from a first end of said nozzle to a first portion of a skin surface of a subject; and (b) at least one camera configured for recording one or more images of the first portion of the skin surface, optionally wherein the one or more images comprise optical or digital images. In some embodiments, the device further comprises a signal processing unit (e.g., a microprocessor or computer) configured to analyze the one or more images and to calculate one or more measurements related to an indentation in the first portion of the skin surface. In some embodiments, the signal processing unit is configured to calculate an edema score based on the one or more measurements. In some embodiments, the signal processing unit comprises a microprocessor. In some embodiments, the device comprises a housing comprising an opening for the first end of the nozzle and an opening for one or more lens of the one or more camera. In some embodiments, the housing comprises a triggering mechanism. In some embodiments, the device is portable and/or hand-held. In some embodiments, the device is configured to communicate (e.g., wirelessly communicate) with another device (e.g., a computer or hand-held electronic device) and/or includes a display, e.g., an LED screen) to display images, data and/or an edema score.

The distance of the first end of the nozzle and/or the lens of the at least one camera from the skin surface of the subject can be varied, e.g., to improve consistency of skin surface indentation and/or quality of the images obtained by the camera. Typically, when the pulse pressure is held constant, the farther away the nozzle from the skin surface, the shallower the indentation for pulse. Accordingly, in some embodiments, it can be useful to have the nozzle as close to the skin as possible to provide the deepest indentation. In some embodiments, the distance of the first end of the nozzle and/or the lens of the at least one camera can be varied depending upon which area of skin surface is being assessed for edema, e.g., the ankle, leg, foot, hand, wrist, arm, chest, etc. In some embodiments, the first end of the nozzle is positioned between about 0.2 inches and about 1.5 inches from the skin surface of the subject (e.g., about 0.2, 0.3, 0.4, 0.5, 0.6, 0.7, 0.8, 0.9, 1.0, 1.1, 1.2, 1.3, 1.4, or about 1.5 inches from the skin surface of the subject). In some embodiments, the first end of the nozzle is between about 0.5 inches and about 1 inch or between about 1 inch and about 1.5 inches from the skin surface of the subject. In some embodiments, the first end of the nozzle is about 1 inch from the skin surface of the subject.

The angle of the nozzle and/or the camera lens can also be varied, (e.g., depending upon the skin surface being used (e.g., ankle, leg, arm, back, etc.) and/or other conditions, such as, but not limited to pressure, ambient light level, and expected level of edema severity, in order to obtain the most consistent measurements, and/or camera images that are more easily analyzed (e.g., that have the highest level of contrast between indented and non-indented skin surface). Typically, angling the nozzle provides an elliptically shaped indentation, which, in some embodiments, provides a more defined boundary between indented and non-indented skin surface on one side of the indentation. For example, in some embodiments, such as when an additional light source is used to direct light to the skin surface, placing the nozzle and lighting at an angle (e.g., from the camera lens and/or from the skin surface) can help with light deflection in the camera images. In some embodiments, having the nozzle and light directed straight down to the skin surface does not result in an indentation with an edge highlighted as much as when the nozzle and light are at an angle.

In some embodiments, the one or more pulses of air are directed to the skin surface of the subject such that the pulse is approximately perpendicular to the skin surface (i.e., the angle of incidence of the pulse on the skin surface is approximately 0 degrees (°)). However, in some embodiments, the pulse or pulses are directed to the surface of the skin at an angle. For example, in some embodiments, one or more pulses of air are directed to the skin surface of the subject at an angle of incidence between about 45 degrees and about 75 degrees (e.g., about 45, 50, 55, 60, 65, 70 or about 75 degrees). In some embodiments, the angle can be up to 90 degrees. In some embodiments, the one or more pulses of air are directed to the skin surface of the subject at an angle of about 60 degrees. In some embodiments, at least one camera lens is placed so that the lens surface is approximately parallel to the skin surface (e.g., to obtain images of light reflected approximately perpendicularly from the skin surface). In some embodiments, the camera lens is perpendicular to the angle of the pulse or pulses of compressed gas or other air (i.e., the camera lens and the opening of the end of the nozzle are co-planar). In some embodiments, at least one camera lens is placed at an angle to the skin surface and/or to the pulse or pulses from the nozzle.

In some embodiments, the method further comprises illuminating the skin surface of the subject with one or more light source (e.g., to provide improved contrast between indented and non-intended skin surface/or provide directional lighting). In some embodiments, the one or more light source comprises one or more LED lights. In some embodiments, the LED light is a coaxial LED light. In some embodiments, the method comprises illuminating the skin surface with a light while protecting the skin surface from ambient light (i.e., other light present in a room or other environment in which the method is being performed, such as light from a fluorescent overhead light or lights or sunlight from a window). Thus, in some embodiments, the skin surface can be at least partially covered or shaded while the method is being performed to block ambient light from reaching the skin surface. Blocking ambient light while illuminating the skin surface with a particular light source can help to provide more consistent skin surface images and/or indentation area measurements.

In some embodiments, the light source, the nozzle (e.g., the release of the at least one pulse of compressed air or other gas), and the one or more camera are all automatically controlled through a single triggering mechanism. In some embodiments, the triggering mechanism is a switch, button, or trigger located on a housing enclosing a nozzle and a camera. In some embodiments, the triggering mechanism is associated with a touch screen on a device housing or on a separate electronic device, such as a cell phone, tablet computer, laptop computer, or desk top computer (e.g., that can communicate wirelessly with the device components).

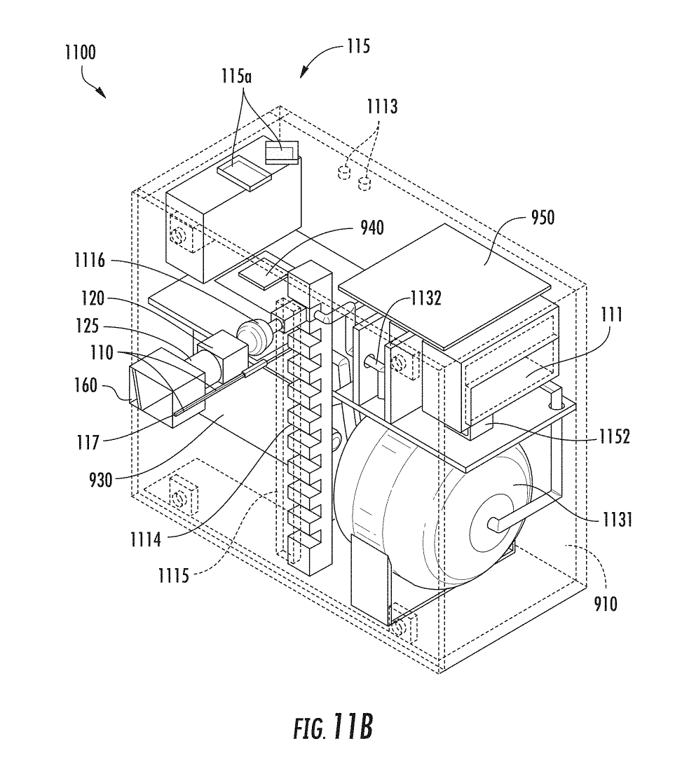

In some embodiments, diffusion lighting can be used to avoid intensity from the bottom of the indentation due to directional lighting. In some embodiments, dark-field lighting (where the light reflected by the skin surface does not enter the camera when no surface deflection presents) can be used. For example, FIG. 10 shows an illustration of a method wherein gas (e.g., air) pulse 1016 from first nozzle end 117 is directed to the surface of the skin of a subject. Pulse 1016 causes non-indented first portion of skin surface 135a to form indented first portion of skin surface 135b. Incident light 1061 from light source 160 (e.g., a LED light source) is reflected along path 1062 back to light source 160 prior to indentation (i.e., from non-indented first portion of skin surface 135a). After indentation, indented first portion of skin surface 135b reflects light along path 1063 to camera 120.

Thus, in some embodiments, the placement of the camera, light source, and air nozzle can be varied to optimize the capture of the skin's response during the indentation and recovery process. In some embodiments, the light source is positioned to direct a light beam directly at the skin surface (i.e., “straight on” toward the skin surface, where the angle of incidence of the light on the skin surface is about 0°). In some embodiments, the pulse or pulses of compressed air or other gas are coplanar with a beam of light from the light source. Thus, in some embodiments, the pulse or pulses of compressed air or other gas are directed to the skin surface with an angle of incidence of about 0°. Alternatively, in some embodiments, one or both of the light source and the pulse or pulses of compressed air or other gas is positioned to direct the light or the pulse or pulses to the skin surface at an angle. In some embodiments, the camera lens is positioned to capture light directly reflected from the skin surface. Alternatively, in some embodiments, the camera is positioned to capture light reflected from the skin surface at an angle. In some embodiments, the angle between the pulse or pulses and/or the beam of light from the light source and the light reflected from the skin surface and captured by the camera is between about 15 degrees and about 75 degrees or between about 45 degrees and about 75 degrees (e.g., about 45, 50, 55, 60, 65, 70, or about 75°). In some embodiments, the angle between the pulse or pulses of compressed air or other gas and/or the beam of light from the light source and the light reflected from the skin source and captured by the camera is about 60 degrees.

In some embodiments, the method of measuring edema is adapted to measure one or more skin properties of a subject (e.g., elasticity, hydration level, thickness, etc.). Thus, in some embodiments, the method can be adapted for use in dermatology, e.g., with regard to the assessment of various skin disorders or conditions, or in the cosmetics or personal care industry, e.g., to measure the skin effects of cosmetic and/or skin care products. For instance, the method can be adapted to measure potential increased skin moisture levels associated with the use of a skin care product, such as a moisturizer. The method could also be adapted to measure dehydration to determine whether a subject, such as an individual working outdoors, participating in a sport, or performing physical exercise, has lost too much water from the skin, thereby running the risk of suffering the effects of dehydration or accelerated skin aging, and needs to rehydrate.

More particularly, skin hydration level can be related to skin elasticity. When skin becomes less hydrated, it becomes less elastic and can take longer to recover from a pinch or indentation. Similarly, when skin is thicker it can take longer to recover from indentation. Accordingly, in some embodiments, the presently disclosed subject matter provides a method of measuring one or more skin-related property in a subject, the method comprising: (a) directing one or more pulses of compressed air or other gas at a portion of a skin surface of the subject; (b) obtaining one or more images of said portion of the skin surface; and (c) analyzing the one or more images of said portion of the skin surface to calculate one or more of the group comprising, but not limited to, the area of an indentation resulting from contact of the skin surface with the one or more pulses of compressed air or other gas; the depth of indentation resulting from contact of the skin surface with the one or more pulses of compressed air or other gas; the rate at which the indentation resulting from contact of the skin surface with the one or more pulses of compressed air disappears; the maximum depth or area of indentation produced by the pulse or pulses, the length of time or the amount of pressure needed for the indentation to reach a particular depth or area, the amount of time for the indentation depth to fully recover or to recover to a particular percentage of the maximum indentation depth after one or more pulse of compressed air or other gas (e.g., the “recovery time”), the difference in area or depth of indentation produced by pulses of different pressure, and the indentation area as a function of time. The measurement calculated by the analysis can then be compared to measurements taken in standard samples for healthy skin, skin from subjects of different ages, skin of varying degrees of thickness, and/or varying degrees or elasticity or hydration to determine a relative level of a skin property in comparison to the standard samples. In some embodiments, the skin property is selected from thickness, quality, age, hydration level, and elasticity.

Preliminary studies using artificial skin samples used for training medical personal to stage edema are described hereinbelow in Examples 1-3. In these studies, it is shown that the presently disclosed methods and devices can distinguish varying levels of fluid accumulation in edematous tissue samples.

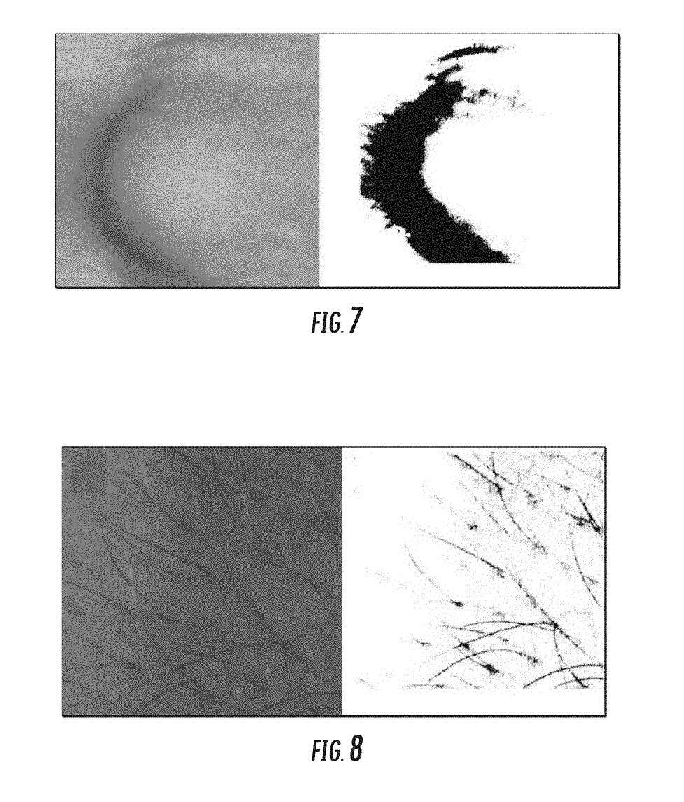

While preliminary studies were generally conducted on samples with a consistent size and color, the use of the presently disclosed methods and devices can be readily extended to, or adapted for, subjects of any age, gender, skin tone, skin hair content/concentration, and body mass index (BMI). For example, skin surfaces with darker skin tones can be wiped with alcohol prior collecting images and/or dark-field illumination techniques (e.g., as described hereinabove) can be used. Dark-field illumination techniques have previously been used commercially to aid in the identification of dents and scratches on dark surfaces of articles of manufacture that can occur during the manufacturing process and can increase contrast between an indented and non-indented skin surface. Alternatively, a correction factor related to skin tone (e.g., measured, for example, using the Fitzpatrick scale) can be input into a signal processing unit to change segmentation parameters and/or use a correction factor during the processing of the camera images. Suitable correction factors can be determined from comparative sample images taken from subjects with different skin tones. The image on the left-hand side of FIG. 7 shows a magnified camera image of a skin indentation in a skin sample with darker skin tone. While indentation is visible in the unprocessed image, processing of the image via threshold segmentation and binarization (see image on the right-hand side of FIG. 7) provides a clear differentiation between indented and non-indented portions of the skin sample.

As another example, preliminary studies with human subjects have indicated that the presence of dark hair in the test area can off-set the baseline measurements of the skin samples. To correct for interference from dark hair, edema measurements can, if possible, be taken from the inside of a subject's wrist or arm or another area where there is generally less hair, or hair can be shaved from a portion of the skin surface of interest just prior to measurement. Alternatively, dark hairs can be labeled in binarized images and digitally removed or otherwise corrected for during image processing. The image on the left-hand side of FIG. 8 shows a camera image of skin surface with dark hair. The image on the right-hand side of FIG. 8 shows a binarized version of the same image after threshold segmentation. The strands of hair are seen as long rods that can be tagged and excluded from analysis during measurement of an area of indentation.

### IV. Devices

In order to address the inconsistencies associated with measuring edema via the pitting method, the presently disclosed subject matter provides, in some embodiments, a device comprising at least one nozzle that can direct a flow or pulse of compressed air (or another gas, such as nitrogen, carbon dioxide, argon, etc.) at the skin surface of a subject and at least one camera for recording one or more images of the skin surface (e.g., after it has been contacted with the flow or pulse of compressed air or other gas). The nozzle can be configured to direct the pulse at a predetermined pressure and/or for a predetermined amount of time. The image or images can be used to more quantitatively determine the effect of the compressed air or other gas on the skin surface (e.g., the size of an indentation (or “dent”) in the skin surface and/or the amount of time needed for the skin surface to recover), thereby providing a more consistent way to measure edema. The device can also be used to measure various skin properties, e.g., elasticity, hydration, etc.

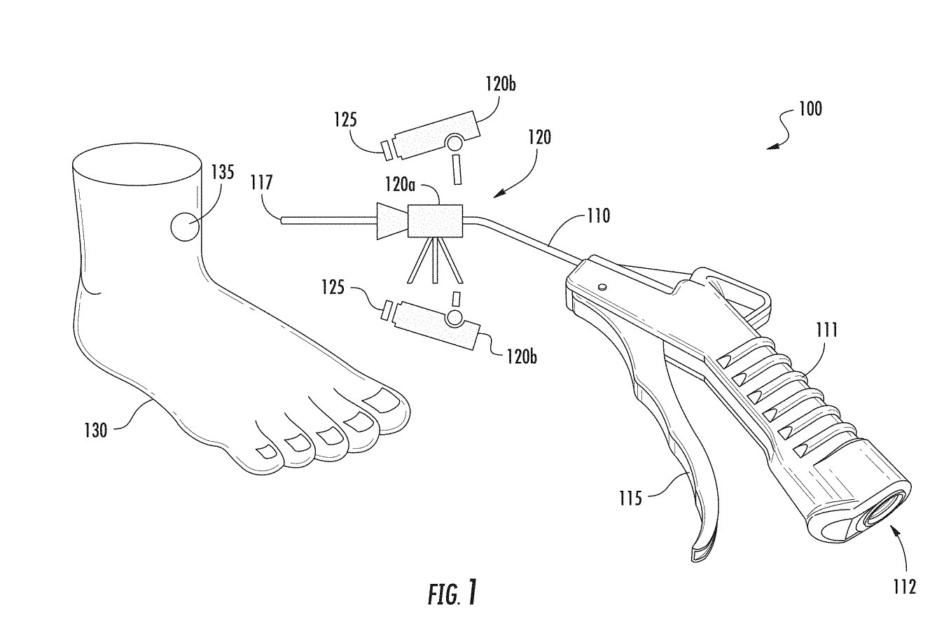

FIG. 1 shows an exemplary device for measuring or monitoring edema (e.g., peripheral edema) and/or other skin properties/conditions. More particularly, FIG. 1 shows device 100 that includes gas nozzle 110 that can be attached to a compressed air/gas line at second nozzle end 112 and that can direct compressed air or another compressed gas at a skin surface (e.g., just above the ankle) of subject 130 who is suffering from or is believed to be potentially suffering from peripheral edema or a condition associated with peripheral edema. First nozzle end 117 is positioned to direct a pulse of compressed air or other gas at first portion of skin surface 135 to produce an indentation. The pulse of compressed air or other gas can be initiated by triggering mechanism 115 (e.g., a trigger that can be manually depressed). Device 100 can be held by handle 111 (e.g., a grip on a molded nozzle body) shown on the right-hand side of the figure. The device of FIG. 1 also includes camera 120, which in this embodiment includes high speed camera 120a and stereo camera 120b having two lenses 125. Camera 120 is configured to collect images of first portion of skin surface 135.

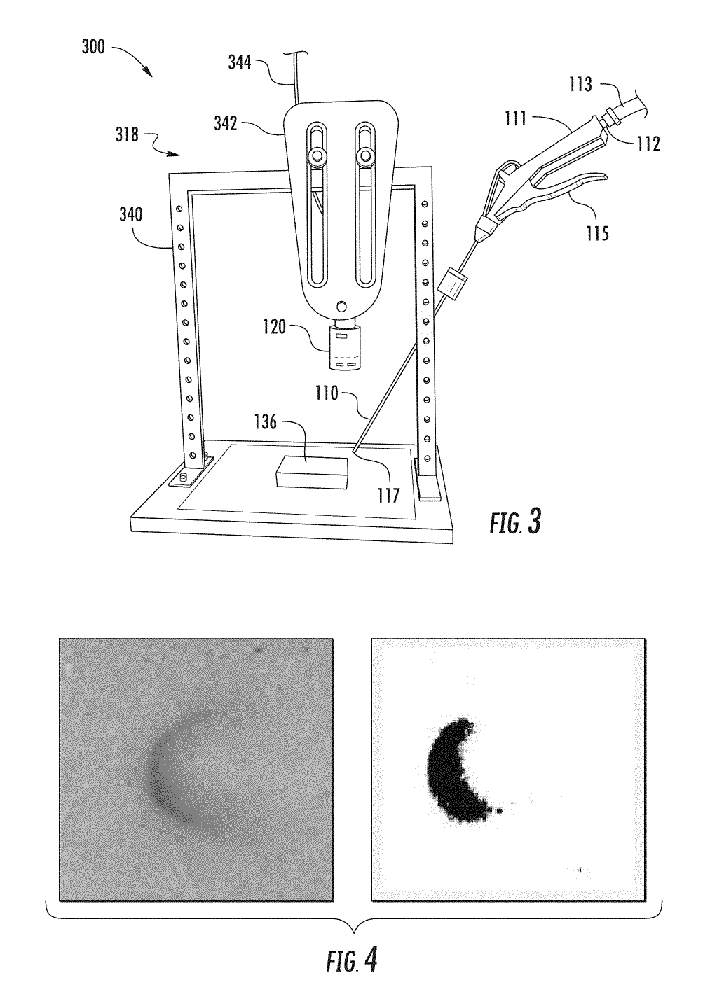

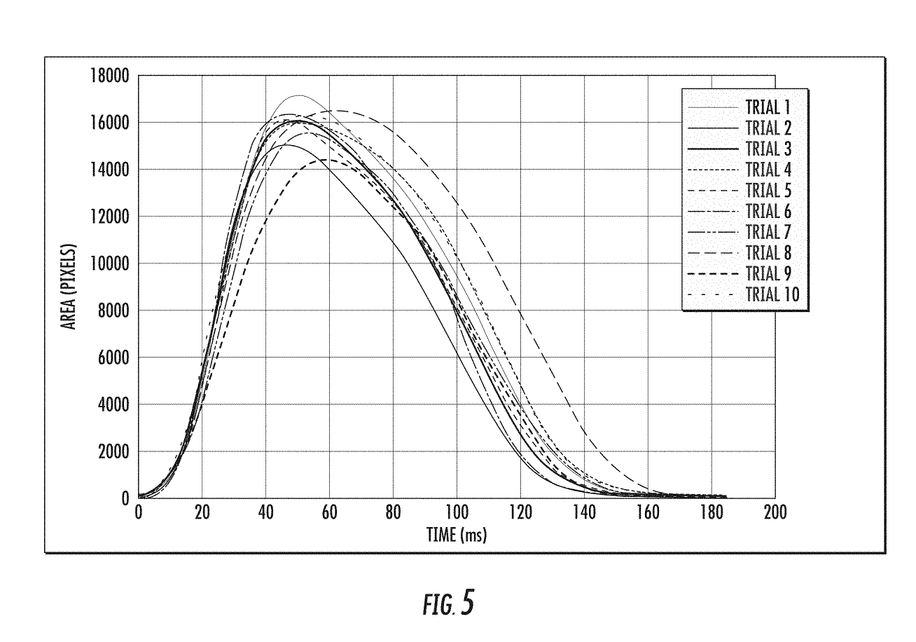

FIG. 3, described hereinbelow in Example 1, shows an alternative device 300 used during preliminary studies of the presently disclosed methods. More particularly, the device of FIG. 3 includes camera 120, which in this embodiment includes a single high-speed camera, configured to obtain images of artificial skin sample 136. Nozzle 110 is configured such that first nozzle end 117 can direct a pulse of compressed air or other gas to the surface of skin sample 136. Unlike in FIG. 1, where first nozzle end 117 is configured to direct the pulse of compressed air or other gas to first portion of skin surface 135 at an approximately 0° angle of incidence, in FIG. 3, first nozzle end 117 is configured to direct the pulse of compressed air or other gas to the surface of artificial skin sample 136 at an angle. Handle 111 and triggering mechanism 115, which, as in FIG. 1, is shown as a trigger that can be manually depressed to initiate release of the pulse of compressed air or other gas from first nozzle end 117, are shown as part of a molded body associated with nozzle 110. Connector 113 (e.g., an air hose) connects second nozzle end 112 to a source of compressed air or other gas, such as a gas tank or a house gas line. Both nozzle 110 and camera 120 are attached to a positioning component 318, which in device 300 includes frame 340 to aid in positioning of the camera and first nozzle end 117, as well as camera holder 342. Camera 120 is attached to frame 340 indirectly via camera holder 342. Camera 120 is also attached, via cable 344, to a signal processing unit, such as computer (e.g., a laptop computer) configured to store and process camera images.

Accordingly, in some embodiments, the presently disclosed subject matter provides a device for measuring edema and/or one or more skin-related properties (e.g., elasticity, thickness, quality, age and/or hydration level), wherein the device comprises (a) a nozzle configured for directing at least one pulse of compressed air or other gas from a first end of said nozzle to a first portion of a skin surface of a subject, and (b) one or more camera configured for recording (i.e., obtaining and storing and/or obtaining and wirelessly or non-wirelessly transmitting) one or more images of the first portion of the skin surface. In some embodiments, the nozzle is a stainless steel, other metal, or plastic nozzle. In some embodiments, the nozzle can have a diameter of about ⅛ inch. However, other nozzle diameters can be used. In some embodiments, a nozzle with a diameter smaller than about ⅛ inch can be used, e.g., to reduce the amount of compressed air or other gas used to provide the one or more pulses, such as the amount of air needed per pulse. In some embodiments, the device further includes one or more additional device components associated with the nozzle, such as, but not limited to, a gas source, tubing to connect the nozzle to a gas source, a gas pressure monitor, a device or system for regulating gas pressure, and a triggering mechanism to manually or electronically initiate a pulse or pulses to be expelled from the nozzle. Manual triggering mechanisms can include, but are not limited to, buttons, triggers, switches, and the like.

Any suitable camera or cameras can be used, including, but not limited to, a stereo camera, a video camera, a high-speed camera, and combinations thereof. In some embodiments, the camera can obtain a series of images over a predetermined period of time (e.g., over about a one second time span or over the course of one or more minutes (e.g., 1, 2, 3, 4, 5, 6, 7, 8, 9, or 10 minutes). In some embodiments, the camera is automatically triggered to start obtaining images when the compressed air or other gas is released from the nozzle. Thus, the camera can be a suitable high speed commercially available camera, such as, but not limited to IDS model UI-3140CP “Rev 2” USB (IDS Imaging Development Systems, GmbH, Obersulm Germany). In some embodiments, the camera can be fitted with a magnifying lens. For example, a high speed camera lens and/or adapter can be used in combination with the camera, e.g., to provide high magnification pictures and/or to provide a suitable frame rate. For instance, suitable commercially available camera magnification lenses include the Kowa LM25JC ⅔″ 1 Megapixel 25 mm lens (Kowa American Corp., Torrance, Calif.). The camera can also include circuitry or a communications component to transmit camera images to a computer or other device that can analyze the data.

The device can also include one or more additional components as described hereinbelow, such as, but not limited to, a battery or other power system (e.g., a power cord), a microprocessor, a communication component (e.g., a transceiver or transmitter for wireless communications or wiring to connect the camera or a device-associated microprocessor to another computing device (e.g., a laptop or desktop computer), a positioning component (e.g., a hand or foot hold, a cuff, straps, a positioning arm or guide) and a light source.

In some embodiments, the nozzle and/or the camera, and/or one or more additional device component are provided in one or more housing unit. In some embodiments, the housing unit comprises a thermoplastic or thermosetting polymer casing. In some embodiments, the housing unit can comprise a metal casing or casing comprising a combination of polymer and metal.

In some embodiments, the device of the presently disclosed subject matter comprises a single housing unit to hold and/or position individual components of the device. In some embodiments, the housing can hold one or more cameras and at least one nozzle. In some embodiments, the housing further holds a microprocessor. In some embodiments, the housing further holds a battery. In some embodiments, the housing further holds a gas source, such as a gas cartridge or a gas compressor, optionally in combination with a gas tank.

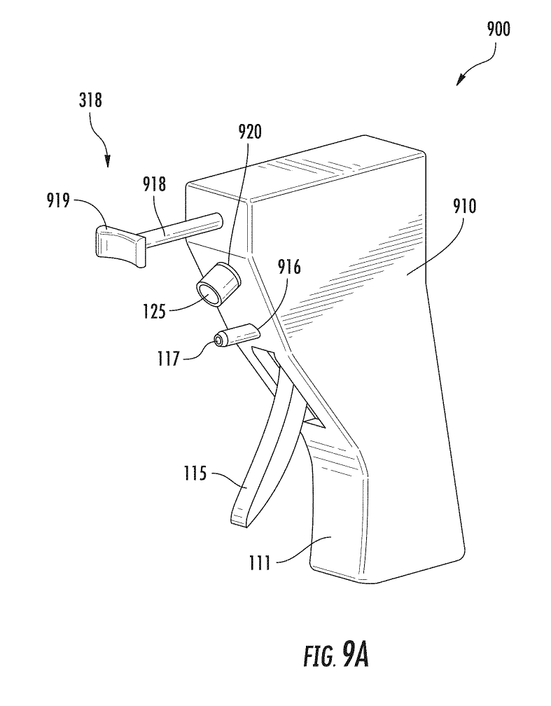

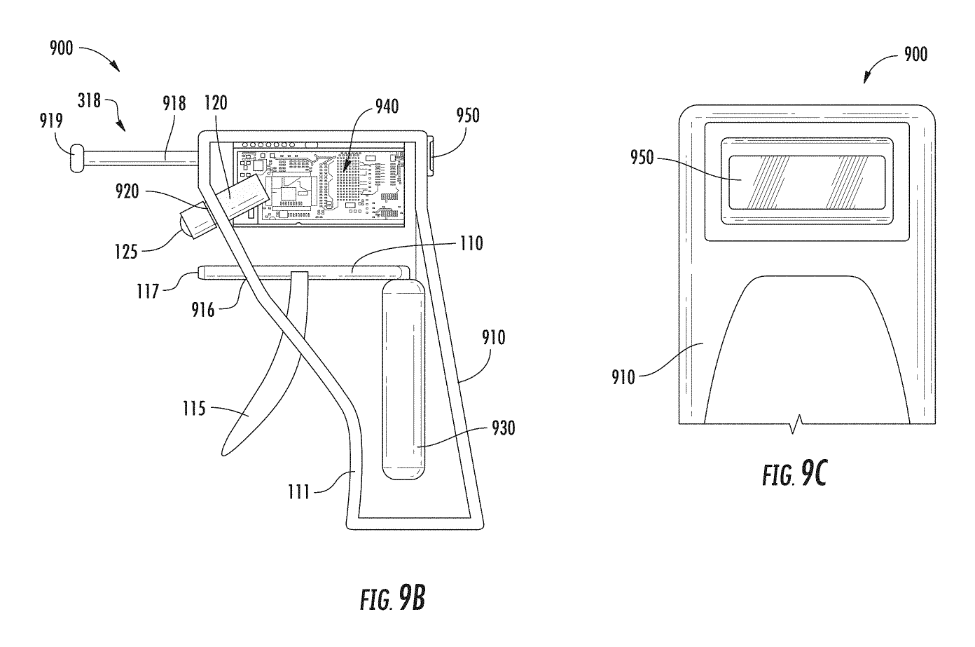

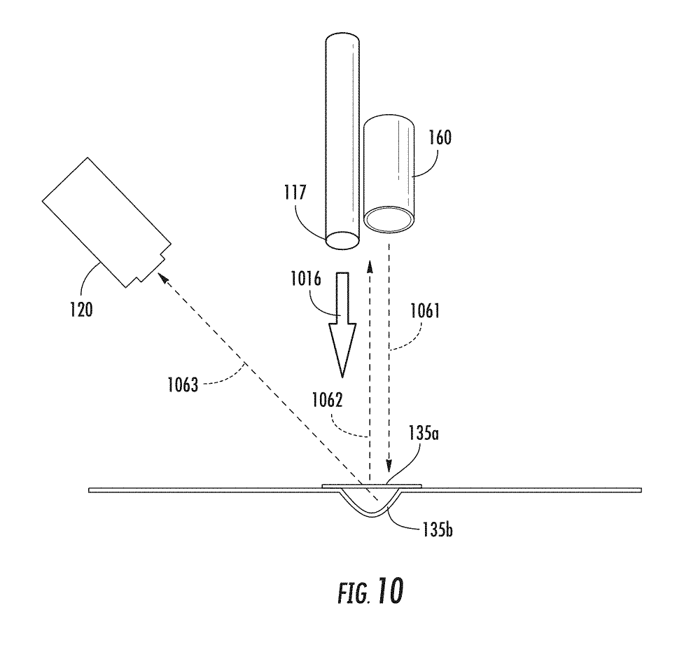

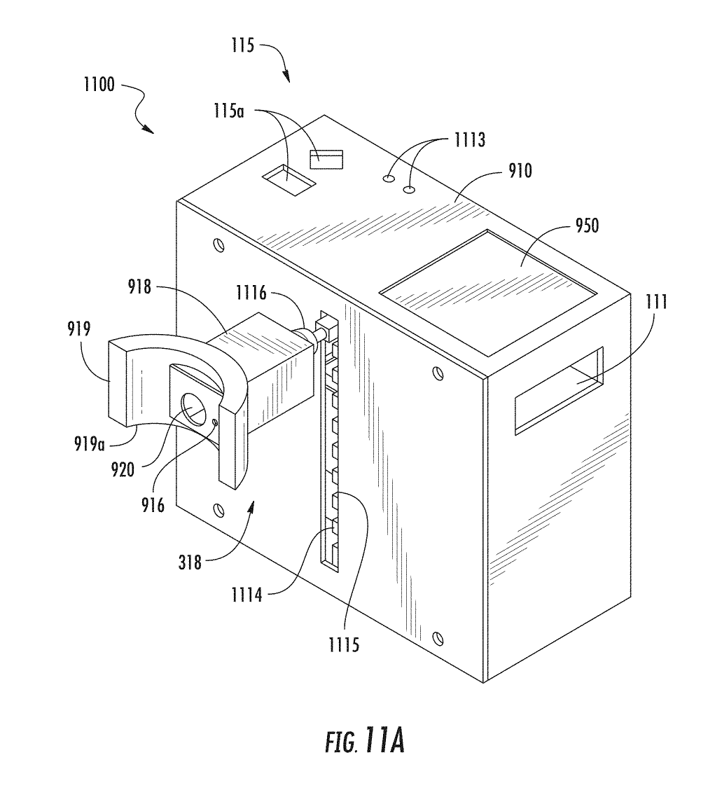

FIGS. 9A-9C show exemplary hand-held device 900 of the presently disclosed subject matter comprising a single housing unit. Referring to FIGS. 9A and 9B, device housing 910 has opening 920 for camera lens 125, wherein opening 920 is configured such that camera 120 can obtain images of a portion of a skin surface impinged by a pulse or pulses of compressed air (or another gas) directed at the skin surface from nozzle 110. Device housing 910 can also contain opening 916 for first nozzle end 117. In some embodiments, nozzle opening 916 can be configured to aid in positioning first nozzle end 117 of nozzle 110 at a predetermined distance from and/or angle to the skin surface.

While not shown in FIGS. 9A and 9B, in some embodiments, the device can have multiple openings for multiple camera lenses, such that images can be obtained of an indented area on the skin from multiple distances and/or angles. Alternatively, one opening can be configured such that multiple camera lenses can be positioned in the same opening to take images of the indented area from multiple distances and/or angles. In some embodiments, the device could also have multiple nozzle openings, for example, configured to direct pulses of gas (e.g., compressed air) at the skin surface from multiple distances and/or angles, either simultaneously or sequentially.

Returning to FIGS. 9A and 9B, in some embodiments, device housing 910 further comprises positioning component 318 which includes positioning arm 918 with end 919. Positioning arm 918 can extend from a front surface of device housing 910 (i.e., the same side of the housing as opening 920 and opening 916). During use of device 900, end 919 of positioning arm 918 can be contacted to the subject (e.g., at a portion of skin surface near the skin surface to be contacted by the pulse of compressed air or other gas), thereby positioning first nozzle end 117 and camera lens 125 at predetermined distances and/or angles from the portion of skin to be contacted with the compressed air or other gas. Thus, positioning arm 918 can be used to provide for increased consistency in the distance between the end of the nozzle and the skin surface from measurement to measurement. In some embodiments, end 919 of positioning arm 918 can include a curved surface configured to fit around the surface of the portion of skin to be assessed (e.g., of an arm or leg of an adult human subject). In some embodiments, positioning arm 918 can be adjustable, so that the nozzle and/or lens distance and/or angle can be adjusted based on the portion of the skin surface being tested, the size of the subject, and/or to test the same skin area using different predetermined distances and/or angles.

In some embodiments, the housing further contains a compressed air or other gas source (e.g., a cylinder and piston for compressing air, a table top air compressor, or a gas cylinder, tank, or cartridge), wherein the compressed air or other gas source is configured in flow communication with the nozzle and is also configured such that manual application of pressure on a triggering mechanism, such as a trigger or button, by an individual using the device (e.g., a health care provider or the subject him or herself) releases a pulse of compressed air (for example, of a predetermined pressure) or other gas from the source and out the end of the nozzle. For example, as shown in FIGS. 9A and 9B, device housing 910 includes triggering mechanism 115, which in this embodiment is configured as a trigger extending from an opening in device housing 910. As shown in FIG. 9B, triggering mechanism 115 is configured to initiate the release of a pulse of air from first nozzle end 117. Nozzle 110 is also in flow communication with gas source 930, which in this embodiment is shown as a gas cartridge which can hold compressed air or another gas (e.g., CO₂, helium, nitrogen, argon, or a mixture of compressed gases). The gas cartridge can be replaceable (e.g., via a door in device housing 910) or rechargeable. As an alternative to including a gas source inside the housing, the housing can include an inlet for receiving compressed air or another gas (e.g., from an air compressor or gas tank external to the housing) such that the inlet provides flow communication of the compressed air or other gas to the nozzle and wherein pressure on the trigger initiates the release of a pulse of compressed air or other gas.

In some embodiments, such as that shown in FIGS. 9A-9B, the housing can be configured to comprise handle 111, which in this embodiment is a grip that is a molded portion of device housing 910 configured to fit in a human hand. Triggering mechanism 115 can be configured near handle 111 so that the device can be held and used with a single hand. Alternatively, triggering mechanism 115 can a button, switch, or dial in the device housing that can be pressed, rotated or otherwise moved to initiate the flow or pulse (or a series of pulses) of compressed air (or other gas), i.e., instead of the trigger shown in FIGS. 9A and 9B. In some embodiments, all or a portion of device housing 910 can comprise a thermosetting or thermoplastic polymer.

As further shown in FIG. 9B, device 900 can include signal processing unit 940 encompassed within device housing 910, which in this embodiment is a microprocessor configured to extract and/or manipulate data from one or more images obtained/recorded by camera 120. For example, signal processing unit 940 can be configured to process camera images and calculate indentation area, the change in indentation area over time (e.g., a rate of indentation and/or indentation recovery), indentation depth, and/or an edema score calculated from indentation area or other data extracted from an image or images from camera 120. In some embodiments, signal processing unit 940 is configured to calculate or measure data related to one or more topological features of the indentation (e.g., circumference, etc.). In some embodiments, signal processing unit 940 includes a communications component (e.g., a wireless communications component, such as a transceiver) that can communicate data and/or camera images to a separate computer or other electronic device. In some embodiments, the communication can be wireless (e.g., Bluetooth). In some embodiments, signal processing unit 940 is configured to store and/or compare data from previous measurements. In some embodiments, as shown in FIGS. 9B and 9C, device 900 can include display window 950, e.g., on the back (or side) of device housing 910, that can display data related to indentation measurements and/or an edema score calculated based thereon.

In some embodiments, the device can also include one or more batteries (e.g., rechargeable batteries), enclosed within the housing. The housing can include one or more detachable sections that can be removed to access and/or replace the batteries. Alternatively, the device can be configured to be plugged into a power source (e.g., an electrical outlet or charging station) to be charged prior to use or during use.

In some embodiments, the device can include at least one light source (e.g., a visible or white light source, such as a bright field light source or a light emitting diode (LED); an infrared light source; etc.) to illuminate the portion of the skin surface being indented by the compressed air or other gas. In some embodiments, the device housing can include one or more openings or transparent areas associated with the light source or sources contained within the housing, such that light from the light source or sources can be directed toward the skin surface being indented. The light source can provide enhanced and/or more even/consistent illumination of the skin surface, thereby providing better quality images of the skin surface and more accurate data measurement based on the images. The light source can also provide more consistent conditions from measurement to measurement.

As an alternative to the trigger-containing, gun-like device of FIGS. 9A-9C, the presently disclosed device can also be configured as a pen-like hand held device. For instance, such a device could comprise a cylindrical or rectangular cuboid housing containing an air or other gas cartridge, a gas (air) tube, a gas (air) nozzle, and a camera. In some embodiments, the housing can also contain a light (e.g., a LED light). The device can further contain a transmitter or transceiver for wireless (e.g., Bluetooth) communication of data from the camera to a display screen, computer, cell phone and/or other hand-held electronic device (e.g., a tablet computer). Additionally or alternatively, the device can include a signal processing unit (e.g., a microprocessor) within the hand-held housing.

For example, in some embodiments, a pen-like device can be configured such that an air nozzle is placed about 1-2 inches from a front end of the device housing within a recessed opening in the housing and a camera and a LED light are positioned above the nozzle at an angle (e.g., a 45° angle). On the front side of the housing, a hole can provide an outlet for the pulse from the nozzle so that it can make contact with the skin surface of a subject, and so that the camera can to take images of the skin surface where the pulse makes contact and so that the light (if present) can illuminate the skin surface. During use, the front side of the housing can be placed directly on the skin of the subject and a button on the top or side can be pressed to activate release of a pulse of gas (air) and to activate the camera (as well as the light, if a light is present). The back of the housing can include a plug-in to refill the gas (air) cartridge, as well as to recharge the device.

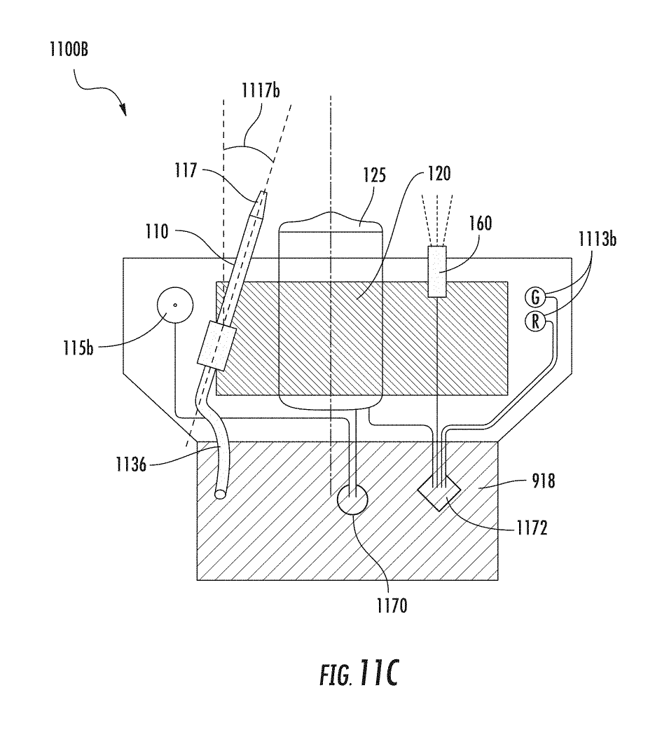

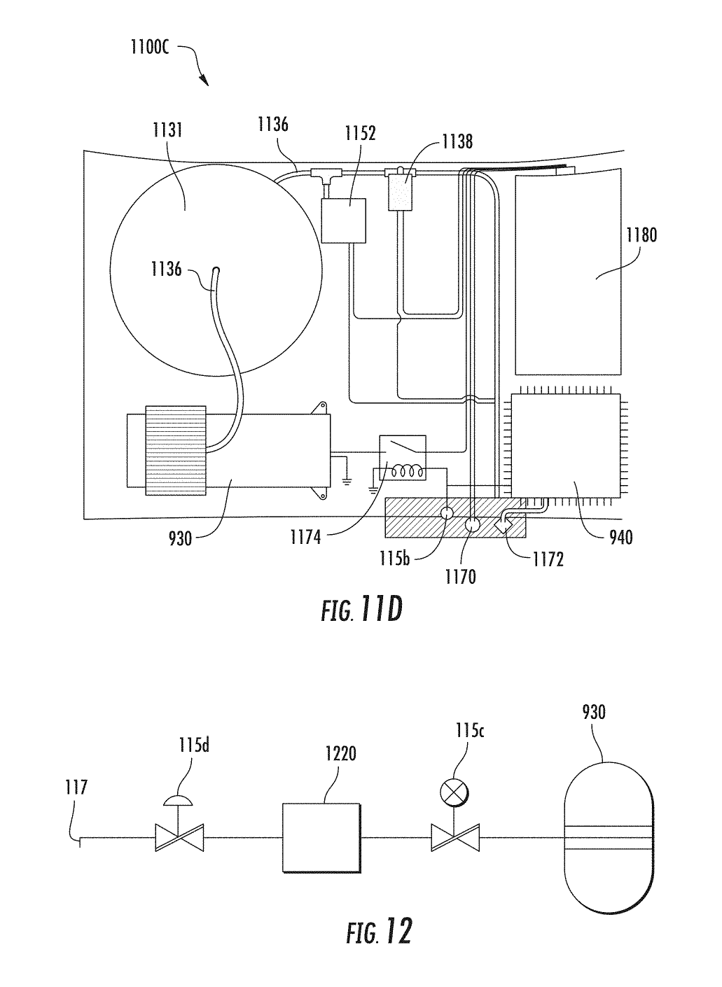

FIGS. 11A and 11B show an alternative embodiment of a device of the presently disclosed subject matter. Device 1100 is an example of a portable, box-like device comprising device housing 910 configured to be stationary while in use. As shown in FIG. 11A, the top of device housing 910 can include triggering mechanism 115, including control (on/off) buttons 115a (e.g., for controlling the release of a pulse or pulses of air by device 1100 and/or for controlling one or more cameras contained in device 1100, and/or one or more light sources (e.g., LED light sources) to illuminate a skin surface during edema measurement with device 1100). The top of device housing 1110 can also include indicator lights (e.g., LED indicator lights) 1113 (e.g., to indicate if a device component is in use and/or if the component is in need of maintenance) for different components (e.g., the camera or the nozzle) of device 1100, as well as display window 950 for displaying edema measurements or data obtained by device 1100. One side of device housing 910 can include handle 111, which in this embodiment is an indentation in the side of device housing 910. Handle 111 can be used as an aid in moving and/or positioning device 1100. The opposite side of device housing 910 can include a second indentation to serve as a second handle.

As further shown in FIG. 11A, to one side of device housing 910 is positioning component 318, which includes positioning arm 918 with end 919 configured to form cuff 919a, ball and socket joint 1116, adjustment track 1114, and opening 1115. Positioning arm 918 is attached to the main body of device 1100 at opening 1115 and can be moved vertically via adjustment track 1114 inside device housing 910 and partially visible through opening 1115. End 919 of positioning arm 918 is configured to form cuff 919a, which can be used to help indicate to an individual using the device where to position the limb of a subject being assessed for edema and/or to secure the limb in position. For example, device housing 910 can be placed on the ground and a subject can place his or her leg or ankle in contact with cuff 919a. Alternatively, device housing 910 can be placed on a table or counter and the subject can place his or her arm in cuff 919a. In the center of cuff 919a is opening 920 for a camera lens of a high-speed camera contained within positioning arm 918 and opening 916 for a first end of a nozzle contained within the device. Opening 920 can be configured to protect the lens of a camera inside positioning arm 918 from ambient light. For example, the camera lens can be recessed inside opening 920. Opening 920 can also be configured to include a light source or sources (e.g., a LED light source) to illuminate the skin surface being assessed. While opening 920 and opening 916 can be separate openings, e.g., as shown in FIG. 11A, the openings can be combined to provide a single opening for both the nozzle and camera.

FIG. 11B shows a front perspective view of device 1100 with device housing 910 shown as transparent. As in FIG. 11A, the top of device housing 910 of device 1100 includes control buttons 115a of triggering mechanism 115, indicator lights 1113, and display window 950. One side of device housing 1110 includes handle 111. Signal processing unit 940 (e.g., a microprocessor) is shown within the upper portion of device housing 910, under control buttons 115a and indicator lights 1113. Gas source 930 (e.g., an air compressor) is near the bottom of device housing 910 and is in flow communication with air tank 1131. Pneumatic valve 1132 can be used to maintain a steady pressure of air to first nozzle end 117 of nozzle 110. Pressure monitor 1152 (e.g., a pressure regulator) is also included in device housing 910. FIG. 11B shows that inside positioning arm 918 shown in FIG. 11A is ball and socket joint 1116, which attaches the positioning arm to the main body of the device and provides adjustability to the device, e.g., in combination with vertical adjustment via adjustment track 1114 inside opening 1115. Within the positioning arm is also camera 120 (e.g., a high-speed camera) with camera lens 125, nozzle 110, and light source 160 (e.g., a coaxial LED light source). The combination of the vertical track and the ball and socket joint provide for height and/or angle adjustment of the camera and nozzle openings of the device so that it can be used to measure edema in a sitting or standing subject in an at home or clinical environment, as well as to provide the ability to measure edema in different locations in a subject limb and/or in subjects of different limb length and/or circumference.

FIG. 11C shows a top view of elements of a positioning arm in an alternative embodiment of device 1110 of FIGS. 11A and 11B, i.e., device 1110B. Nozzle 110 is attached to air hose 1136 which is in flow communication with a gas source not shown in the figure. First nozzle end 117 can direct a pulse of compressed air or other gas toward a subject in front of device 1110B at an angle 1117b. Camera 120 with camera lens 125 is next to light source 160 (e.g., a coaxial LED light source). Positioning arm 918 further includes wiring 1170 for a camera battery and wiring 1172 for transmitting camera images to a microprocessor in the main body of device 1100B. Wiring 1172 is also attached to indicator lights 1113b at the top of positioning arm 918. The triggering mechanism of device 1110B can include pressure switch 115b for controlling an air compressor within the device and is present on the top of positioning arm 918.

FIG. 11D is a bottom view of elements inside an alternative embodiment of device 1100 of FIGS. 11A and 11B, i.e., device 1100C, showing additional details of the internal components of the main body of the device. More particularly, air tank 1131 is in flow communication with gas source 930 (e.g., an air compressor) and with pressure monitor 1152 (e.g., a pressure regulator) and air controller 1138 (e.g., a solenoid-controlled 2/2 way plunger valve) via air tubes (e.g., hoses) 1136. Relay 1174 connects pressure switch 115b and signal processing unit 940 (e.g., a microprocessor) to gas source 930. Signal processing unit 940 and battery 1180 are also attached to other components via wiring 1172 and 1170, respectively.

The box device of FIGS. 11A-11D includes an automated compressed gas delivery system using an on-device air compressor as the source of the compressed air. An air tank included in such a system can act as a reservoir for compressed air. Suitable air tanks and air compressors for use in the device of FIGS. 11A-11D and/or other table-top and/or portable devices of the presently disclosed subject matter include, but are not limited to, the Viair 0.5 Gallon Tank (Viair Corporation, Irvine, Calif., United States of America) and the Viair C-Model 100C air compressor (Viair Corporation, Irvine, Calif., United States of America). Any suitable commercially available air hose or hoses (e.g., rubber hoses) can be used to connect different components of the compressed gas delivery system of the presently disclosed devices. For example, a suitable hose can be a rubber hose having an inner diameter of about ⅜ inch. The air hose can be customized, as necessary to have a suitable length to connect various components (e.g., the air tank and the air compressor; the air tank to an air pressure monitor, and/or an air pressure monitor to an air nozzle) of any particular device. The hoses can also include suitable ports/fittings to connect the various components. In some embodiments, one or more union tee or other hose connector fitting can be used to connect the hoses.

In some embodiments, for automated air pressure monitoring, as in the device of FIGS. 11A-11D, the presently disclosed device can include a digital air pressure monitor. Having a precise digital air pressure monitor can also increase the repeatability of the pulse of compressed air or other gas and the resulting skin indentation. The monitor can electronically signal the microprocessor when the desired air pressure is reached. In some embodiments, a pneumatic valve can be incorporated in the compressed air delivery system to aid in the automated delivery of a quick controlled burst of air. The pneumatic valve can be electronically signaled by the microprocessor to deliver the desired burst of air at the appropriate time. In some embodiments, the pneumatic valve is a solenoid valve.

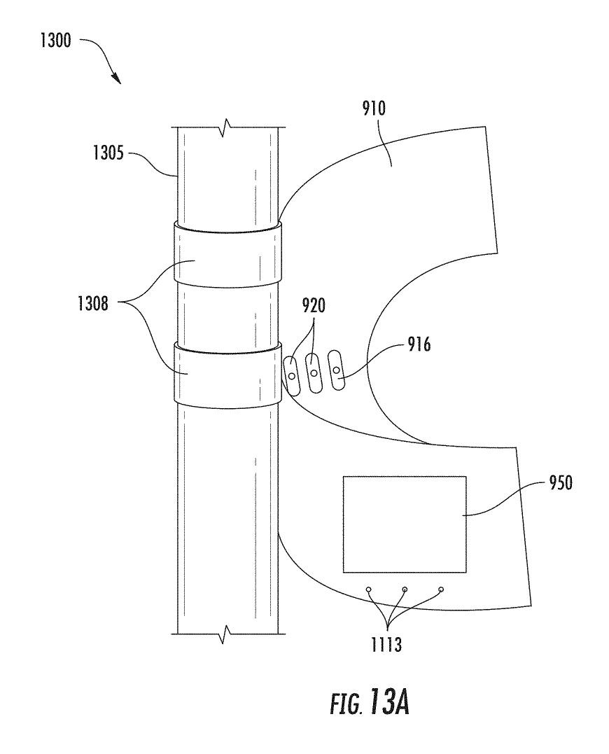

FIG. 12 also shows an exemplary unit for standardizing gas pulses in the presently disclosed devices. While the unit is described for compressed air, the system can be used for other compressed gases. For example, FIG. 12 shows gas source 930, such as an air compressor or an air (gas) tank connected to an air compressor or to a facility air or gas line). Gas source 930 is connected to volumetric space/chamber 1220 (e.g., in an air hose and/or an air nozzle) via pressure gage/switch or regulator 115c, which can be part of a triggering mechanism of a device and which can ensure a constant and consistent pressure pulse release from a volumetric space/chamber 1220. The size of volumetric space/chamber 1220 can be designed such that the pressure of the air or other gas pulses reduces by no more than about 1% during use Release of air (or other gas) from volumetric space 1220 to first nozzle end 117 is controlled via nozzle switch 115d. In some embodiments, nozzle switch 115d is controlled by a microprocessor or computer, e.g., to help insure that each pulse of air or other gas has the same volume.

Returning to FIGS. 11A-11D, the size of box device 1100, 1100B, and/or 1100C is not particularly limited. However, to enhance the portability of the device, the dimensions of the box device can be, in some embodiments, between about 12 inches and about 24 inches high, between about 6 to 12 inches wide, and between about 12 and about 28 inches long. In some embodiments, the device can be about 12.5 inches high, about 6.5 inches wide and about 14.5 inches long. While box-like devices comprising gas tanks and compressors can weigh more than hand-held devices containing replaceable or rechargeable gas canisters or cartridges or devices attached to in-house gas lines, in some embodiments, a box-like device such as that shown in FIGS. 11A-11D can still weigh less than about 15 pounds or less than about 10 pounds.

While box device 1100, 1100B, and 1100C of FIGS. 11A-11D is shown in an arrangement where a subject limb is intended to be placed at a side of the main housing, as an alternative embodiment, a box-like device can be designed in another orientation, e.g., such that the main body of the device (including the air tank, compressor, microprocessor and battery) is configured in a box placed on a floor or counter and having a foot or hand rest on the top of the main housing, indicating a position for a subject in need of edema assessment on a portion of the arm or leg to place a foot or hand. The camera, gas nozzle and light (if present) can be contained in an adjustable arm extending upward from the main box (either directly upward from the main box or out of a side of the box and then upward), with openings for the nozzle, camera lens and light (if present) positioned facing an area over the top of the box where a subject's arm or leg would be positioned. A triggering mechanism, such as one or more control buttons, can be placed on the top of the adjustable arm or main box.

In some embodiments, the device of the presently disclosed subject matter can be configured and/or designed for in-home use, e.g., so that a subject suffering from or at risk of edema can use the device to monitor edema (e.g., peripheral edema) on a regular or as needed basis at home. To facilitate the in-home use of the device, it can be advantageous, in some embodiments, to configure the device for use in combination with an assistive device already in use by the subject, such as a cane, crutch, wheelchair or walker, or with a similar component (e.g., an extension/stabilizer arm) particularly provided to the subject to make the use of the presently disclosed edema measurement device easier for a subject who can otherwise have trouble using the device (e.g., due to an unrelated pre-existing or edema-related physical impediment limiting the subject's range of motion). For example, peripheral edema is often related to edema in the ankle, and it can be difficult for some subjects to bend over to a sufficient degree to effectively position a hand-held device, such as that shown in FIGS. 9A-9C, to measure edema.

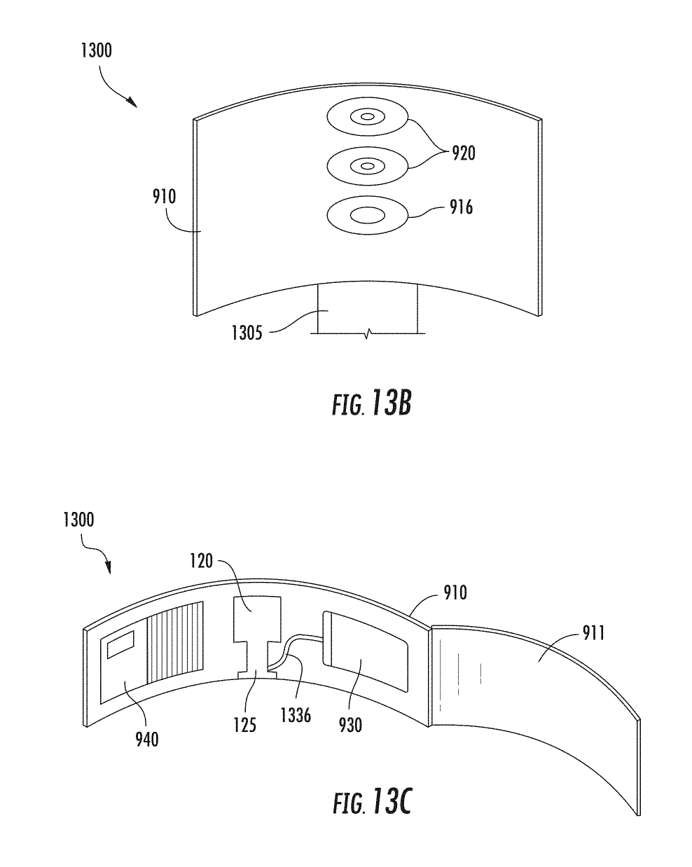

FIGS. 13A-13C show schematic drawings of an exemplary device of the presently disclosed subject matter suitable for use in combination with an assistive device. More particularly, FIG. 13A shows a side perspective view of device 1300, comprising a device housing 910 configured in a curved “C”-shaped or cuff-shaped form and attached via adjustable straps 1308 to assistive device 1305 (e.g., a cane, crutch, or a leg of a walker). In some embodiments, straps 1308 can comprise fabric or plastic material with Velcro® strips to fasten one section of a strap to another to hold device housing 910 in place. Device housing 910 can comprise a thermosetting or thermoplastic material shaped to fit around the calf or ankle of a human subject (e.g., an adult human subject). On the inside of the center of the cuff are openings 920 for two camera lenses or for a camera lens and a light, as well as opening 916 for the end of a gas nozzle. Display window 950 and indicator lights 1113 can be positioned on the outer surface of one side of device housing 910.

FIG. 13B shows a front view of device 1300, looking directly toward the inside of housing 910. Assistive device 1305 is visible behind housing 910. Openings 920 and opening 916 are visible in the center of housing 910.

FIG. 13C shows a top view of housing 910 of device 1300 with cover 911 open to reveal components inside housing 910. Gas source 930 (e.g., an air or gas cartridge) is connected to an opening (e.g., opening 916 of FIGS. 13A and 13C) under camera lens 125 on the center inside of device housing 910 via tubing 1336. Camera 120 is attached to camera lens 125. Camera 120 can include, for example, two cameras or a single camera. If camera 120 is a single camera, device 1300 can include a light source under or over the camera, positioned to illuminate a skin surface through one of openings 920 shown in FIGS. 13A and 13B. Signal processing unit 940 (e.g., a circuit board and/or microprocessor) is also located in device housing 910 to enable analysis and/or automation of the edema measurements. Control (e.g., on/off switches) for the device can be included on the top of the housing or the housing can include a communications component configured to receive a wireless signal, e.g., from a remote control or hand-held personal electronic device (e.g., a smart phone or tablet computer) to initiate measurement using the device. The communications component can be included as part of signal processing unit 940 as the place of signal processing unit 940. Alternatively, display window 950 of FIG. 13A can include a touch screen configured to initiate pulse release and control the camera and/or light when the subject or another individual touches the touch screen.

In an alternative embodiment, the cuff-shaped device of FIGS. 13A-13C can be configured so that straps 1308 can be used to position housing 910 around a subject's ankle or calf. In such embodiments, when the device is fitted nearer the subject's skin, the device can include a LED or other light on the inside of the cuff to illuminate the skin surface being indented in order to improve the quality of the images obtained by the camera or cameras.

In some embodiments, a two-piece device can be provided, e.g., wherein components of the device are enclosed in two housing units. For example, the two-piece device can include at least one housing unit configured to be hand-held. In some embodiments, the hand-held unit can include the outlet of the nozzle for directing at least one pulse of compressed air or other gas to the skin surface of a subject and a camera or cameras for recording one or more images of the skin surface. In some embodiments, the hand-held unit can also include a light to illuminate the skin surface, optionally in combination with a cover to block ambient light from illuminating the skin surface being indented by the pulse or pulses of compressed air or other gas. The second unit can generally remain stationary during use of the device and/or can contain a signal processing unit (e.g., including a microprocessor) to analyze images from the camera or cameras and/or calculate measurements related to an indentation in a skin surface impinged by the pulse or pulses of compressed air or other gas. In some embodiments, the second unit can be attached to a wall (e.g., in an exam room in a hospital, clinic or doctor's office) so that it can be more easily or directly attached to a house compressed gas (air) line or compressed gas (air) tank used to supply compressed gas (e.g., air) to the nozzle in the hand-held unit. However, in some embodiments, the second unit is not attached to a wall or is detachable attached to a wall or other fixture and can be moved between measurements (e.g., to relocate the device closer to a subject or to move the device from room to room). In some embodiments, the second unit can contain a compressed air or other compressed gas canister or cartridge, a gas (air) tank, and/or a gas (air) compressor. In some embodiments, the two units can be connected by tubing to provide air or other gas to a nozzle in the hand-held unit from the second unit and/or to contain electronic circuitry/wiring to control the release of the pulse or pulses from the nozzle, to control the camera, and/or to transmit data from the camera to a microprocessor in the stationary unit. In some embodiments, the hand-held unit includes a gas cartridge.

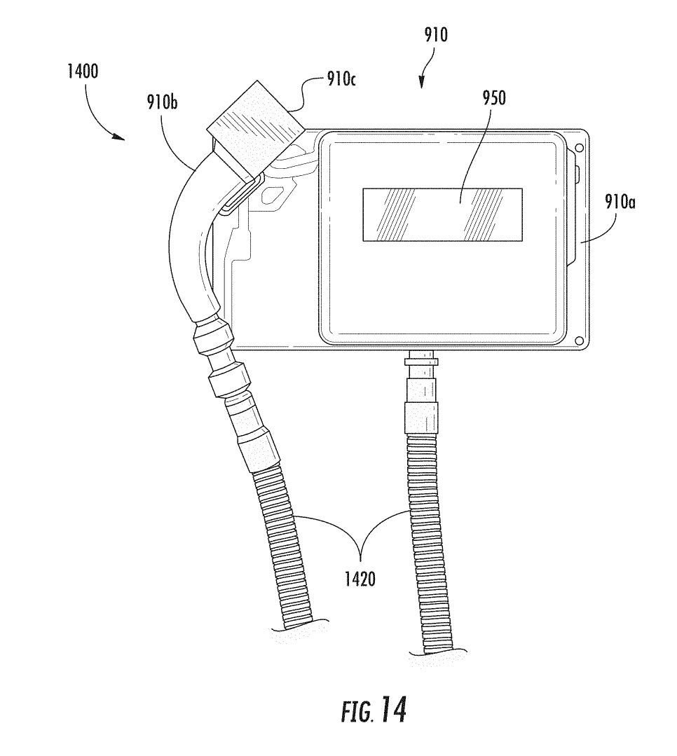

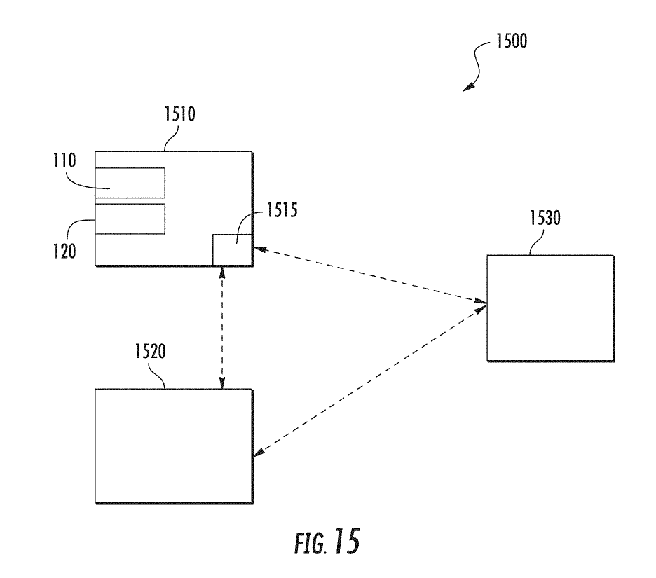

FIG. 14 shows an illustration of exemplary device 1400 comprising device housing 910 configured as two housing units, housing unit 910a and housing unit 910b, which further includes outlet 910c. Housing unit 910a (i.e., a “second housing unit” or a “stationary housing unit”) can include a signal processing unit to provide edema measurements to display window 950. Housing unit 910a can also include a gas cartridge or an outlet for connection to an in-house gas line or a gas tank. Housing unit 910a is connected by tubing 1420 to hand-held unit 910b. Hand-held unit 910b can be manually moved to a site on a skin surface of a subject to be assessed for edema. At the end of hand-held unit 910b is outlet 910c which can contain openings for a gas nozzle, one or more camera lens, and one or more light. These openings can be recessed inside outlet 910c so that the outer edges of the terminus of outlet 910c can overhang these openings. Thus, when outlet 910c is placed on the skin surface of a subject, the end/tip of the gas nozzle and the camera lens(es) are positioned at a suitable distance (e.g., about 0.2 to 1.5 inches) from the subject's skin surface for providing the pulse or pulses of compressed air or other gas to the skin surface and for recording images of the resulting skin indentation. The overhang of outlet 910c can also block ambient light to provide more consistent lighting conditions for the images. The end of outlet 910c as shown in FIG. 14 is round, however, the end of the outlet can be any suitable shape, e.g., round, oval, square, rectangular, triangular, hexagonal, etc.

Alternatively, in some embodiments, the two units are not physically connected. In some embodiments, the stationary unit contains a transceiver to collect data and/or to send signals to control components in the hand-held unit via wireless communication (e.g., Bluetooth or WiFi communication), and the hand-held unit can contain a transceiver or transmitter-receiver to transmit data to the stationary unit and to receive signals from the stationary unit. In some embodiments, the hand-held unit can comprise an outlet to connect to an in-house gas line or to a gas tank to supply compressed air or another gas to the nozzle.

Thus, in some embodiments, the presently disclosed subject matter provides a device for measuring edema (e.g., peripheral edema) and/or one or more skin-related properties. By way of example and not limitation, the one or more skin-related properties can include elasticity, thickness, quality, age, or hydration level. In some embodiments, the device comprises: (a) a nozzle for directing at least one pulse of compressed air or other gas from a first end of said nozzle to a first portion of a skin surface of a subject; and (b) at least one camera for recording one or more images of the first portion of the skin surface. In some embodiments, the device can include at least two or more nozzles and/or at least two or more cameras. In some embodiments, at least one camera includes at least two lenses.

In some embodiments, the device further comprises a housing configured to contain or partially contain one or more of the other components of the device. In some embodiments, the housing can be formed from a thermoplastic or thermosetting polymer. In some embodiments, the polymer can be selected based on its cost, rigidity, weight, and/or a combination of such factors. For example, in some embodiments a low cost, low weight, relatively rigid polymer can be used to prepare the housing. The housing can comprise a first opening for the first end of the nozzle and at least a second opening for one or more lens of the at least one camera. In some embodiments, the housing can comprise one or more additional openings, e.g., for one or more additional nozzle or camera lens(es).

In some embodiments, the device can further comprise a positioning component including one or more component of the group comprising, but not limited to, a frame for the attachment of one or more other device components (e.g., the nozzle and/or the camera), a positioning arm, a cuff, a strap or belt, a foot hold, and a hand hold. In some embodiments, the device comprises positioning component comprising a positioning arm. In some embodiments, the positioning arm extends from a main body of a device (e.g., of a device housing) and an end of said positioning arm is configured to be placed directly adjacent to a second portion of the skin surface of the subject. In some embodiments, the end of the positioning arm is configured to position the first end of the nozzle at a first distance and/or a first angle from the first portion of the skin surface of the subject and/or to position one or more lens of the at least one camera at a second distance or distances and/or a second angle or angles from the first portion of the skin surface of the subject. In some embodiments, the housing can comprise one or more additional positioning arms or other positioning components.

In some embodiments, the device is configured to attach to an assistive device, such as a walker, cane, crutch, wheelchair, etc. used by the subject. In some embodiments, the device is provided with an extension arm (e.g., a telescoping arm) or support that can be attached to allow a subject to more easily position the device at a skin surface area that can otherwise be hard to access.

In some embodiments, the device housing further comprises a handle or hand grip. In some embodiments, the housing comprises a triggering mechanism, comprising, for example, a trigger or button configured to initiate the release of one or more pulse of compressed air or other gas from the first end of the nozzle and/or the recording of one or more images by the at least one camera when the trigger or button is pushed. In some embodiments, the triggering mechanism comprises a sensing mechanism that triggers the release of one or more pulse and/or the recording of one or more images. For example, the sensing mechanism can be a mechanism that senses the presence of a subject positioned for edema measurement using the device, e.g., via a motion detector or using body heat from the skin surface or the presence of the skin surface in the field of vision of the camera. The triggering mechanism can also be voice activated or electronically activated, e.g., via remote communication from a computer or other electronic device. In some embodiments, a single triggering mechanism automatically initiates both pulse release and image recordation.

In some embodiments, the device can further comprise a signal processing unit (e.g., contained within the housing) to analyze one or more images and/or calculate one or more measurements related to an indentation in the first portion of the skin surface caused by contact of said first portion of the skin surface with the at least one or more pulses of compressed air or other gas. In some embodiments, the signal processing unit can be programmed to calculate one or more of a rate of indentation, a depth of indentation, an indentation area, a change in indentation area as a function of time, and/or a measurement related to one or more topological features of the indentation. In some embodiments, the signal processing unit can be programmed to analyze a plurality of images recorded sequentially over a pre-determined period of time after said first portion of the skin surface is contacted with the one or more pulses of compressed air or other gas. In some embodiments, the signal processing unit can further calculate the change in indentation area in the first portion of the skin surface as a function of time. In some embodiments, the signal processing unit is configured and/or programmed to calculate an edema score from one or more measurements related to the indentation, such as a change in indentation area as a function of time. In some embodiments, the edema score is calculated as a rating of severity of edema on a numerical scale. In some embodiments, the scale can be a scale of 1.00 to 4.00, i.e., to correlate to a traditional edema severity measurement based on manual pitting.

However, the presently disclosed subject matter is not limited to such an edema scale. Other, potentially more robust scales can be used, e.g., depending upon the more particular application of the method and/or statistical analysis of the data.

In some embodiments, the signal processing unit can comprise a microprocessor. By microprocessor is meant a particular structure, namely a multipurpose, clock-driven integrated circuit that can include both integer and floating point arithmetic logic units (ALUs), control logic, a collection of registers, and scratchpad memory (e.g., cache memory), linked by fixed bus interconnects. The control logic fetches instruction codes, and initiates a sequence of operations required for the ALUs to carry out the instruction code. Any suitable microprocessor can be used, such as, but not limited to commercially available microprocessors from Intel (Santa Clara, Calif., United States of America), Qualcomm (San Diego, Calif., United States of America), Samsung (Seoul, South Korea), Advanced Micro Devices (Sunnyvale, Calif., United States of America) and the like. In some embodiments, data processing using the microprocessor can involve defining a sequence of algorithm operations in a computer language, such as, but not limited to, MatLab or C++. In some embodiments, using the microprocessor can comprise defining a sequence of algorithm operations in one computer language to provide a source code and using a commercially available compiler (such as the Intel C++ Compiler) to generate machine code (i.e., sometimes termed object code) from the source code. In some embodiments, the device comprises an on-board computer, e.g., an ARM-based Linux computer with USB ports and a LED touch screen.

In some embodiments, the device (e.g., the housing of the device) can comprise one or more display windows for displaying one or more measurements related to indentation and/or an edema score. In some embodiments, the device can further comprise a data storage unit for storing one or more measurements related to an indentation and/or one or more edema scores. In some embodiments, the data storage unit is present in the device housing. The data storage unit can store measurements/edema scores obtained at different times of the same day, week or month; using different portions of the skin surface of the same subject; and/or obtained using different subjects. In some embodiments, the display window can display current data and prior data from the same subject simultaneously. In some embodiments, the display window and/or the data storage unit are part of the signal processing unit.

In some embodiments, the device (e.g., the signal processing unit or another part of the device) includes a communications component to transmit optical or digital images and/or related measurements or edema scores to another device, such as, but not limited to, a cell phone, a portable computing device (e.g., a tablet computer or laptop computer), a desktop computer, a server computer, a persistent storage device, and/or a network. In some embodiments, the communications component is configured to communicate wirelessly. In some embodiments, the communications component comprises a transceiver or a transmitter-receiver.

In some embodiments, the device further comprises a source of compressed air or other gas attached to a second end of the nozzle or a channel or tube for connecting an external source of compressed air or other gas to the second end of the nozzle. The source of compressed air or other gas can comprise, but is not limited to, a table top air compressor or a compressed gas (e.g., carbon dioxide) dispenser, tank, or cartridge. In some embodiments, the device can further comprise a battery compartment and/or one or more batteries to provide power to the device. In some embodiments, the battery can be a rechargeable battery. In some embodiments the housing comprises a battery compartment and/or one or more batteries. Alternatively, in some embodiments, the device comprises a power cord, e.g., to plug the device into an electrical outlet in a house, office or hospital.

In some embodiments, the device can comprise one or more light sources configured to direct light toward the first portion of the skin surface. In some embodiments, the light source is a LED light source. In some embodiments, the light source is a coaxial LED light source. In some embodiments, the light source is present in a device housing. In some embodiments, the housing can include one or more openings or transparent areas configured to allow one or more beams of light from one or more light sources contained within the housing to exit the housing.

In some embodiments, the device is portable. In some embodiments, the device includes at least one hand-held housing unit.

### V. Systems

In some embodiments, the presently disclosed subject matter provides a system for measuring and/or monitoring edema and/or one or more skin-related properties in a subject. The system can include a device for measuring edema and/or one or more skin-related properties in a subject and a communications device or component to communicate data related to the measurements, e.g., to a local or remote computer or another electronic device. Thus, the system can be used to communicate edema-related and/or skin property-related data between patients and health care professionals.

The presently disclosed system can comprise a device for measuring edema and/or one or more skin-related property, such as a device as described hereinabove that includes a nozzle configured to direct one or more pulse of compressed air or other gas at a portion of a skin surface of a subject and one or more camera configured for obtaining and/or recording one or more images of the portion of the skin surface. The device can also include a communication component, for communicating data with a mobile electronic device (e.g., a smart phone, a tablet computer, etc.), computer (e.g., desk or laptop computer), or server (e.g., directly and/or indirectly via the mobile electronic device). In some embodiments, the communication is wireless. The communications component can use any type of wireless technology, such as, for example, cellular, Bluetooth, Bluetooth Low Energy (BLE), Wireless USB, WiFi, Near Field Communication (NFC), ZigBee, Mesh Networking, Worldwide Interoperability for Microwave Access (WiMax), Ultra Wideband (UWB) communication, Radio Frequency (RF), Infrared (IR), etc., or any combination thereof. In some embodiments, the communications component comprises a transmitter and/or antenna configured for wireless communication. In some embodiments, the communications component comprises a transceiver or a transmitter-receiver.

In some embodiments, the system comprises a device for measuring edema and/or one or more skin-related property, such as a device as described hereinabove, that is designed to be an in-home and/or portable device used by the subject themselves or by a non-medically trained individual (e.g., a caretaker or family member of the subject) present in the same location as the subject, that can transmit edema and/or skin property measurements and/or related data (e.g., optical or digital images obtained from a high speed camera of the device) to a remote site, such as a hospital, clinic, or doctor's office so that medical personnel, such as the subject's physician and/or other members of the subject's health care team, can monitor the subject remotely in order to determine if the subject needs to be seen in person and/or if the subject's treatment regimen needs to be changed or discontinued. For example, if the transmitted data indicates that the subject's edema score is increasing, the subject's physician can schedule an in-person appointment with the subject and/or increase the dosage of a medication (such as a diuretic) being administered to the subject to decrease and/or control the edema. If the edema is under control, the subject can avoid unnecessary doctor visits and/or the doctor can decrease the amount of or eliminate a medication or other treatment being administered to the subject. Thus, the presently disclosed subject matter relates, in some aspects, to telemedicine applications. Telemedicine, which is the use of telecommunications technology to deliver medical information or services to patients or other users at a distance from the provider, is a rapidly growing field of clinical medicine. The local device can transmit data to a remove device via the internet or another network or using cellular technology.

In some embodiments, the device for measuring edema and/or one or more skin-related property (which can be portable or non-portable) is present in a treatment room (e.g., in a clinic, hospital or doctor's office) with the subject and a medical professional. The device can be configured to transmit edema related measurements or data to remote electronic devices (e.g., a computer server) via a cellular network and/or the internet directly or indirectly via one or more electronic devices, such as a cellular phone, a PDA, a docking station, personal computer, or other computing device with communications capability. Thus, in some embodiments, the device can transmit edema-related measurements or data (and/or skin property-related measurements or data) to a local device such as a desktop computer or docking station located in the treatment room or a mobile electronic device (e.g., a smart phone, a personal digital assistant (PDA), a tablet computer or a laptop computer) located in the treatment room, which can then transmit data to a remote server, computer, or storage device.

FIG. 15 shows a schematic illustration of exemplary monitoring and measurement system 1500. Device 1510 (i.e., a device for measuring edema and/or one or more skin-related property) be provided as part of a system 1500 with one or more local (i.e., present at the same location as device 1510) computing/electronic device 1520 (e.g., a smart phone, a personal digital assistant (PDA), a tablet computer, a laptop computer, a personal computer (PC), etc.), and/or one or more non-local server computer or persistent storage device 1530. For example, device 1510 can be a portable device that can be taken from one examining room to another in a medical clinic, hospital or doctor's office or can be an in-home device, present at the residence or work place of the subject. Device 1510 includes nozzle 110 configured to direct one or more pulses of compressed air or other gas at a portion of the skin surface of a subject, one or more camera 120 (e.g., comprising a high-speed camera and/or one or more stereo cameras), and communications component 1515. Communications component 1515 is configured to communicate (e.g., wirelessly communicate) with local device 1520 and/or remote device 1530. In some embodiments, communication component 1515 is configured to communicate with both local device 1520 and remote device 1530. In some embodiments, communication component 1515 communicates wirelessly to local device 1520 and local device 1520 communicates to remote device 1530 (e.g., via the internet or a cellular network).

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Device Set-Up

A mechanical indentation device for measuring edema was set up as shown in FIG. 3. Device 300 includes high speed camera 120 equipped with a macrolens and compressed air nozzle 110 with first nozzle end 117, both fixed to 16×13.5 inch frame 340 of positioning component 318 for consistent camera and nozzle angle and distance. High speed camera 120 is attached to frame 340 using holder 342. Nozzle 110 is connected to an external pressure source at second nozzle end 112 by connector 113 which comprises tubing having an inner diameter of 0.25 inches. During use, the tubing is allowed to fill completely with pressurized air (120 pounds per square inch (psi)) to provide a consistent volume of air (5.8 cubic inches). Trigger 115 on handle 111 of nozzle 110 is then squeezed and the puff is released from first nozzle end 117 to indent artificial skin sample 136. For studies using live subjects, an adjustable holder can be added to the device to keep a subject limb still and to maintain a constant distance from the nozzle tip. High speed camera 120 records the skin response (<1 second) at a frame rate of 400 frames per second and is connected via wiring 344 to a computer for direct storage and processing of the image files.

### Example 2

### Data Collection

Preliminary studies using the device described in Example 1 were completed using Lifeform® skin samples (American 3B Scientific, Tucker Ga., United States of America), which are used to train clinicians on pitting edema. The Lifeform® skin samples include four individual pieces that represent the four levels of pitting edema severity (1+, 2+, 3+, and 4+). Each Lifeform® sample was tested five times by two different individuals under the following conditions; 1) nozzle angle of incidence (45° and 60°), and 2) distance from the sample (0.5, 1, and 1.5 in). All testing was completed with an overhanging fluorescent light. The testing protocol provided for determination of the setup configuration that provided optimal results (the best ability to distinguish different skin samples).

For each testing scenario, the compressed air was first released on the skin sample via a metal nozzle by hand-squeezing the blowgun trigger. Simultaneously, the process of the indentation and the re-bounce of the sample caused by the compressed air was captured by the high-speed camera in the form of video at a frame rate of 400 frames per second. The measurement takes less than 10 seconds to complete.

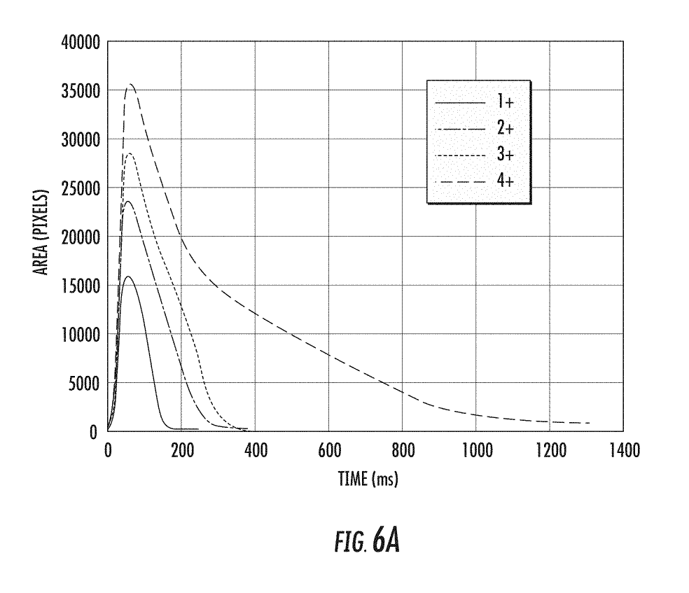

The captured video was processed in MATLAB. The video file read into MATLAB was first separated into individual image frames. Next the images are cropped to focus on the indentation. See FIG. 4, left. After properly cropping, each individual frame was then changed into a black-white image binarized per a greyscale threshold. See FIG. 4, right. This threshold is automatically calculated by using the graythresh( ) function in MATLAB. To accommodate for AC lighting conditions, the code generates an average threshold level of the first twenty images in the video file. Once completed, the final threshold for image processing is set to 83% (5/6) of that average threshold. By lowering the threshold, only the desirable darkest parts of the image, the air-induced indentation, are coded black allowing the indentation to be “seen”.

After the black/white binarization, the number of black pixels in each image was calculated and used as the size of the indentation as the result of the compressed air “puffing”, and a plot of the number of black pixels versus time was generated. The starting and ending of the indentation-recovery process were determined by using the average dark-area of the first five frames as the baseline: the first image frame with the dark area size greater than that average is considered the starting and the last frame with area greater than the average is considered the end. Based off the indentation area vs. time curve, the maximum indentation area, rebound time, and area under the curve were calculated.

### Example 3

### Edema Severity Classification

The mean and standard deviation of the “area under the curve (or AUC)” and the maximum indentation area (or MIA) were utilized as measures to evaluate how a certain trial can be classified to one of the edema levels (1+ to 4+). AUC is the numerical integration of the indentation size over time that consequently reflect both the magnitude of the indention and the time duration of the skin response to the compressed air puffing. Mathematically, AUC is defined:

AUC=Σₖ₌₀^{M}Pk (4)

where pₖ is the number of dark pixels in the k-th image frame and M is the number of frames that show the skin/tissue response from the starting to the ending point; MIA is defined as:

MIA=max(pₖ), k∈{1, 2, . . . , M} (5)

For the ten (or eight for the one with two outliers removed) trials of each of the four edema levels, a “mean” response was first obtained, whose AUC value are MAUCⱼ, j∈{1,2,3,4}. The “AUC distance (D_{AUC})” of an unknown trial i, i ∈{1,2, . . . , 40} to edema level j is defined:

D_{AUC}_{ᵢ}^{j}=AUCⁱ−MAUCⱼ (6)

The same approach was applied to the “MIA distance” measure (D_{MIA}), where:

D_{MIA}_{ᵢ}^{j}=MIAᵢ−MMIAⱼ (7)

After the distances to all four levels are compared, an unknown trial i is classified to its closest edema level. i.e.,

Trial i→Edema j (8)

where j=arg j min D_{AUC}_{ᵢ}^{j}. And

Trial i→Edema j, (9)

where j=arg j min D_{MIA}_{ᵢ}^{j}.

Quality of images from various air nozzle angle and distance was inspected and it was determined that the angle of 60° and distance of 1″ produce the best images. Results analyzed below are from images with this configuration.

Repeatability study results from the ten trials for edema level +1 shown below in FIG. 5. Two trials (#2 and #8) were removed from the trials shown in FIG. 5 due to human errors involved with those two trials (the operator believed the amount of time the trigger was held significantly differs from other trials). Despite these outliers, a visual inspection of FIG. 5 shows that there was a tight spread over the data from all other trials.

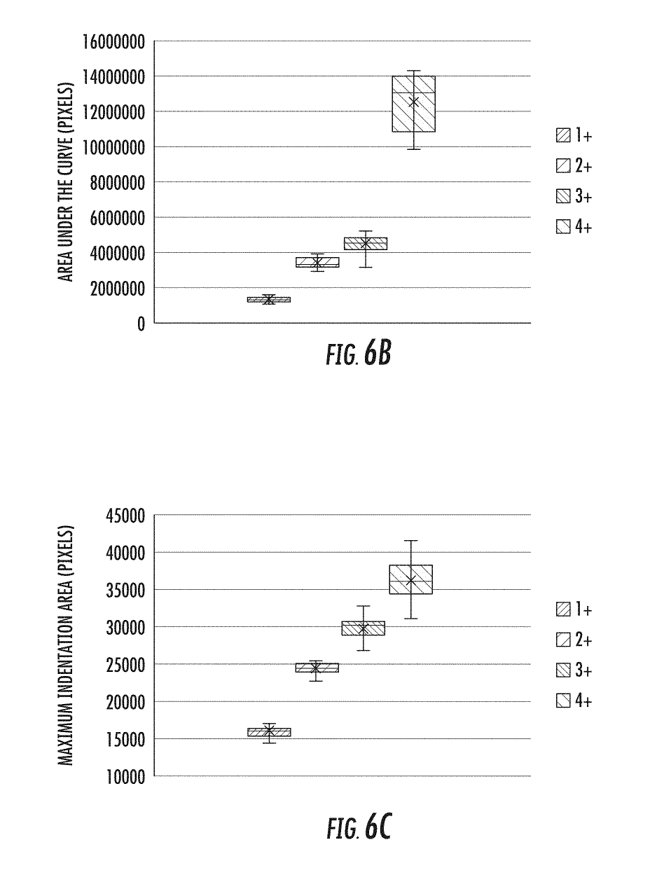

To test the system's ability to distinguish between edema levels, each skin sample representing a different level of edema was tested with multiple trials. The averages of the trials for each level are plotted as in FIG. 6A, which shows a visible difference between the four levels of edema, especially between peak values (which is the maximum area, related to the depth of indentation) found roughly around 50 milliseconds, as well as the amount of time for each sample to fully recover from the indentation.

FIGS. 6B and 6C show more quantitively the device's ability to distinguishing edema severity. FIG. 6B includes AUC results. FIG. 6C shows the same measures but with MIA data, the peak values in FIG. 6A after the four skin samples was puffed by compressed air. The AUC and MIA values are distinct for each level and do not overlap. An unpaired t-test showed statistically significant differences between all levels (p-value ≤0.05). ANOVA found that operator and trial did not have a significant impact on the results indicating a high level of repeatability.

From FIGS. 6A-6C the size of the indentation, which is related to its depth because it is calculated from image darkness, and maximum indentation area both agree with those employed in current medical practice. Based on these results, it appears that when the volume and pressure of the compressed air is controlled, it is possible to classify edema using images captured by a camera.

Using measures D_{AUC} and D_{MIA} to classify the 38 trials, both produced 36 correct and 2 false classifications, yielding ˜95% positive classification. These results demonstrate the potential of using the images to assess edema.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A device for measuring edema and/or one or more skin-related properties, optionally wherein the one or more skin-related properties are selected from elasticity, thickness, quality, age, or hydration level, said device comprising:
(a) a nozzle configured for directing at least one pulse of compressed air or other gas from a first end of said nozzle to a first portion of a skin surface of a subject; and
(b) one or more camera configured to record one or more images of the first portion of the skin surface.

2. The device of claim 1, further comprising a housing, wherein said housing comprises a first opening for the first end of the nozzle and at least a second opening for one or more lens of the one or more camera, optionally wherein said housing comprises a thermoplastic or thermosetting polymer.

3. The device of claim 2, where said housing further comprises a positioning arm, wherein said positioning arm extends from a main body of the housing and wherein, when an end of said positioning arm is placed directly adjacent to a second portion of the skin surface of the subject, said positioning arm controls one or more of the group consisting of a first distance at which the first end of the nozzle is positioned relative to the first portion of the skin surface of the subject; a first angle at which the first end of the nozzle is positioned relative to the first portion of the skin surface of the subject; a second distance or distances at which one or more lens of the at least one camera is positioned relative to the first portion of the skin surface of the subject; and a second angle or angles at which the one or more lens of the at least one camera is positioned relative to the first portion of the skin surface of the subject.

4. The device of claim 2, wherein said housing comprises a handle or hand grip and/or wherein the device is configured to comprise at least one hand-held component.

5. The device of claim 2, wherein said housing comprises a triggering mechanism configured to initiate one or both of the release of one or more pulse of compressed air or other gas from the first end of the nozzle and the recording of one or more images by the at least one camera.

6. The device of claim 1, further comprising a signal processing unit configured to analyze the one or more images and calculate one or more measurements related to an indentation in the first portion of the skin surface caused by contact of said first portion of the skin surface with the at least one or more pulses of compressed air or other gas, optionally wherein each of said one or more measurements is selected from the group consisting of a rate of indentation, a depth of indentation, an indentation area, a change in indentation area as a function of time, and a measurement of one or more topological features of the indentation, further optionally wherein the signal processing unit comprises a microprocessor present in a housing of said device.

7. The device of claim 6, wherein the signal processing unit is configured to analyze a plurality of images recorded sequentially over a pre-determined period of time after said first portion of the skin surface is contacted with the one or more pulses of compressed air or other gas and calculate the change in indentation area in the first portion of the skin surface as a function of time.

8. The device of claim 6, wherein the signal processing unit is further configured to calculate an edema score from the one or more measurements, optionally wherein the one or more measurements comprise a change in indentation area as a function of time, further optionally wherein the edema score is a rating of severity of edema on a numerical scale, such as a scale of 1.00 to 4.00.

9. The device of claim 6, further comprising a display window for displaying one or more measurements related to an indentation and/or an edema score.

10. The device of claims 6, further comprising a data storage unit for storing one or more measurements related to an indentation and/or an edema score obtained at different times of the same day, week or month; using different portions of the skin surface of the subject; and/or obtained using different subjects.

11. The device of claim 1, further comprising a source of compressed air or other gas attached to a second end of the nozzle, optionally wherein the source of compressed air or other gas is selected from one or more of the group consisting of a table top air compressor, a compressed gas canister or cartridge, and a gas tank.

12. The device of claim 1, wherein a second end of the nozzle is attached to a hose or tubing that is detachably connected to a compressed air or other compressed gas source internal to or external to a housing of said device.

13. The device of claim 1, further comprising a gas pressure monitor and/or a device for regulating gas pressure of the pulse of compressed air or other compressed gas exiting the first end of the nozzle.

14. The device of claim 1, further comprising one or more batteries configured to provide power to the device, optionally wherein the one or more batteries are rechargeable batteries.

15. The device of claim 1, further comprising a component configured to wirelessly communicate data from the one or more camera to one or more of a portable computing device, a desktop computing device, a server computer, a persistent storage device, and/or a network.

16. The device of claim 1, further comprising one or more light source configured to provide light to the first portion of the skin surface of the subject, optionally wherein said one or more light source comprises a light emitting diode (LED).

17. The device of claim 16, comprising a positioning arm comprising one or more outlets for the first end of the nozzle, at least one lens of the one or more camera, and light from the one or more light source, optionally wherein the positioning arm has a curved surface and/or wherein the one or more outlets are recessed within the positioning arm to protect the one or more outlets from ambient light.

18. The device of claim 1, wherein said device is portable.

19. The device of claim 1, wherein said device is configured to be attachable to an assistive device, optionally wherein the assistive device is selected from the group consisting of a cane, a walker, a wheelchair, and a crutch.

20. A method of measuring edema in a subject, the method comprising:
(a) directing one or more pulses of compressed air or other gas at a portion of a skin surface of the subject, optionally wherein the portion of the skin surface is a skin surface of an arm, hand, wrist, face, leg, foot, or ankle;
(b) obtaining one or more images of said portion of the skin surface;
(c) analyzing the one or more images of said portion of the skin surface to calculate an area of an indentation resulting from contact of the skin surface with the one or more pulses of compressed air or other gas; and
(d) determining a measure of edema severity in the subject using the area of indentation, optionally wherein the measure of edema severity is provided as a value on a numerical scale, further optionally wherein the numerical scale is between 1.00 and 4.00.

21. The method of claim 20, wherein each of the one or more pulses of compressed air or other gas has a pressure of between about 1 and about 100 psi, optionally wherein the pressure is about 50 psi.

22. The method of claim 20, wherein each of the one or more pulses of compressed air or other gas has a duration of between about 0.1 second and about 30 seconds, optionally wherein the duration is between about 0.2 seconds and about 10 seconds.

23. The method of claim 20, wherein the one or more pulses of compressed air or other gas is a series of pulses, optionally wherein the series of pulses comprises pulses of different pressures, further optionally wherein the series of pulses is directed at the portion of the skin surface of the subject in order from the highest pressure pulse to the lowest pressure pulse or from lowest pressure pulse to highest pressure pulse, thereby providing a pulsed pressure gradient.

24. The method of claim 20, wherein obtaining one or more images comprises obtaining a plurality of images of the skin surface at a series of sequential time points after the skin surface is contacted by the one or more pulses of compressed air or other gas, and wherein analyzing the one or more images comprises analyzing the plurality of images of the skin surface to calculate indentation area as a function of time and/or the maximum depth of indentation.

25. The method of claim 24, wherein the plurality of images comprises at least about 10 images, optionally wherein the plurality of images is obtained over a period of time from about 0.1 second to about 5 minutes.

26. The method of claim 20, wherein the indentation area is calculated as a pixel count of a binarized image of the portion of the skin surface indented by the one or more pulse of compressed air or other gas.

27. The method of claim 20, wherein steps of directing one or more pulses of compressed air or other gas, obtaining one or more images, analyzing one or more images, and determining a measure of edema severity are repeated after one or more minutes, hours, days, or weeks to determine progression or regression of edema in the subject over time.

28. The method of claim 20, wherein the subject is a mammal, optionally wherein the mammal is a human, and/or wherein the subject is pregnant, suffering from a burn or a known or suspected allergic reaction, is being treated for a medical condition with a drug or procedure that can result in peripheral edema, or has been diagnosed with, is suspected of having, or is at risk of developing congestive heart failure (CHF), lymphedema, liver disease, kidney disease, or venous stasis disease.

29. The method of claim 20, wherein the subject has a disease associated with peripheral edema, optionally wherein the disease is congestive heart failure (CHF), and wherein measuring peripheral edema in the subject is performed as part of monitoring the severity of the disease, optionally wherein one or more aspect of the treatment of the subject for the disease is adjusted based on the measure of edema.

30. The method of claim 20, wherein the subject has been diagnosed with or is at risk of lymphedema, optionally wherein the subject is a cancer patient, further optionally wherein the subject is a cancer patient who has had one or more lymph nodes surgically removed and/or who has received radiotherapy.

31. The method of claim 20, wherein the method is performed using a device comprising:
(a) a nozzle configured for directing at least one pulse of compressed air or other gas from a first end of said nozzle to a first portion of a skin surface of a subject; and
(b) at least one camera configured for recording one or more images of the first portion of the skin surface, optionally wherein the one or more images comprise optical or digital images.

32. The method of claim 31, wherein the first end of said nozzle is between about 0.2 inches and about 1.5 inches from the skin surface of the subject, optionally wherein said first end of said nozzle is about 1 inch from the skin surface of the subject.

33. The method of claim 20, wherein the one or more pulses of air are directed to the skin surface of the subject at an angle of incidence between about 45 degrees and about 75 degrees, optionally of about 60 degrees.

34. The method of claim 20, wherein the method further comprises illuminating the skin surface of the subject with light, optionally light from a LED light source.

35. The method of claim 34, wherein the light and the one or more pulses of compressed air or other gas are directed toward the skin surface of the subject at an angle of incidence of about 0 degrees, and wherein the camera is configured to capture light reflected from the skin surface of the subject at an angle of about 45 degrees.

36. The method of claim 34, wherein the illuminating is performed in combination with protecting the skin surface of the subject from ambient light.

37. A system for measuring and/or monitoring edema and/or one or more skin-related properties in a subject, the system comprising:
(a) a device for measuring edema and/or one or more skin-related properties, said device comprising:
(i) a nozzle configured for directing at least one pulse of compressed air or other gas from a first end of said nozzle to a first portion of a skin surface of a subject;
(ii) one or more camera configured for recording one or more images of the first portion of the skin surface; and
(iii) a communications component configured to transmit data, optionally wherein the data is selected from optical and/or digital images collected by the one or more camera; and
(b) at least one of a remote server computer and/or a personal computing device configured to receive data from the device for measuring edema and/or one or more skin-related properties.
